(19) United States
(12) Patent Application Publication
(10) Pub. No.: US 2025/0230418 A1
(43) Pub. Date: Jul. 17, 2025

(54) MODIFIED BATCH AAV PRODUCTION SYSTEMS AND METHODS

(71) Applicant: Ultragenyx Pharmaceutical Inc., Novato, CA (US)

(72) Inventors: Cameron Tage Fulco, West Boylston, MA (US); Jan Thomas Panteli, Arlington, MA (US); Nicholas Alden, Littleton, MA (US); Wei Xue, Burlington, MA (US); James C. Warren, Hollis, NH (US)

(21) Appl. No.: 18/844,595

(22) PCT Filed: Mar. 6, 2023
(86) PCT No.: PCT/US2023/014581

§ 371 Date: Sep. 6, 2024

Related U.S. Application Data
(60) Provisional application No. 63/481,539, filed on Jan. 25, 2023.
(60) Provisional application No. 63/398,355, filed on Aug. 16, 2022.
(60) Provisional application No. 63/317,298, filed on Mar. 7, 2022.

(51) Int. Cl. C12N 7/00; C12N 15/86

(52) U.S. Cl. CPC C12N 7/00; C12N 15/86; C12N 2750/14143; C12N 2750/14152

(57) ABSTRACT

This application provides modified batch systems and methods using perfusion (e.g., intensified perfusion) for increasing productivity and/or cell density in viral particle production to achieve high viral particle, e.g., AAV yield.

### CROSS-REFERENCE TO RELATED APPLICATIONS

This application claims the benefit of and priority to U.S. Provisional Application No. 63/317,298, filed Mar. 7, 2022; to U.S. Provisional Patent Application No. 63/398,355, filed Aug. 16, 2022; and to U.S. Provisional Patent Application No. 63/481,539, filed Jan. 25, 2023, the disclosures of each of which are hereby incorporated by reference in their entireties for all purposes.

### TECHNICAL FIELD OF THE DISCLOSURE

This disclosure relates to modified batch (e.g., intensified perfusion) systems and methods for manufacturing viral vectors such as adeno-associated virus (AAV) vectors, which systems and methods are capable of increasing clinical and commercial grade AAV manufacturing productivity.

### BACKGROUND OF THE INVENTION

Adeno Associated Virus (AAV) is a leading platform for gene delivery for numerous human disease therapies: however, high-dosing requirements for AAV-based therapies and low batch yields in AAV production have resulted in significantly high costs of manufacturing and limited supply to meet patient demand. Despite advances in AAV manufacturing, including development of mammalian producer cell lines usable in suspension culture, there remains significant need to improve existing AAV manufacturing technologies to achieve robust, high-yielding, scalable and cost-efficient processes to meet patient supply needs and reduce the overall cost of treatment using AAV-based therapeutics. Increasing cell density in batch mode alone is not sufficient to increase AAV volumetric yield, and in fact can result in an over 100-fold decrease in yield, e.g., when using a 4-fold increase in seeding density for AAV production. Thus, new systems and methods are needed to enable higher yielding AAV production to meet clinical and therapeutic needs, as well as to reduce the cost of AAV therapy development and clinical and commercial manufacturing.

The present disclosure addresses these needs by providing cost-effective scalable AAV production processes and systems, compatible with mammalian producer cell lines in suspension culture, that reduce cost of goods associated with prior AAV manufacturing platforms.

### BRIEF SUMMARY OF THE INVENTION

This disclosure provides modified batch (e.g., intensified perfusion) systems and methods for AAV manufacturing employing perfusion in AAV producer cell culture (e.g., AAV producer cell suspension culture).

The present disclosure provides modified batch systems and methods using perfusion for AAV production. In some aspects, the disclosure provides systems and methods of producing a recombinant adeno-associated virus (rAAV), comprising the steps of (a) culturing AAV production host cells to a target cell density in a growth vessel using perfusion to supplement the culture with fresh nutrients and/or to remove waste products, wherein the AAV production host cells comprise genetic material encoding one or more AAV components: (b) initiating expression in the AAV production host cells of step (a) of one or more helper virus functions to initiate rAAV production; and (c) culturing the AAV production host cells of step (b) in a production vessel using perfusion to supplement the culture with fresh nutrients and/or to remove waste products, thereby producing the rAAV.

In some embodiments of the systems and methods described herein, the AAV production host cells are insect cells or mammalian cells. In some embodiments, the mammalian cells are Hela cells, Cos-7 cells, HEK293 cells, A549 cells, BHK cells, Vero cells, RD cells, or ARPE-19 cells.

In some embodiments of the systems and methods described herein, the AAV production host cells comprise an AAV producer cell line (PCL)(e.g., a mammalian or insect PCL) comprising genetic material encoding one or more AAV components stably integrated into the AAV production host cell genome. In some embodiments, the AAV PCL may comprise one or more genetic modifications to reduce expression and/or activity of one or more genes and/or proteins to reduce production or accumulation of lactate and/or ammonia.

In some embodiments of the systems and methods described herein, the target cell density in step (a) is at least about 1 E06 viable cells/mL (vc/mL). For instance, the target cell density in step (a) may be between 1 E06 vc/mL to 5 E07 vc/mL. In some embodiments, the target cell density in step (a) is about 1 E06 vc/mL, about 2 E06 vc/mL, about 3 E06 vc/mL, about 4 E06 vc/mL, about 5 E06 vc/mL, about 6 E06 vc/mL, about 7 E06 vc/mL, about 8 E06 vc/mL, about 9 E06 vc/mL, about 1 E07 vc/mL, about 1.1 E07 vc/mL, about 1.2 E07 vc/mL, about 1.3 E07 vc/mL, about 1.4 E07 vc/mL, about 1.5 E07 vc/mL, about 1.6 E07 vc/mL, about 1.7 E07 vc/mL, about 1.8 E07 vc/mL, about 1.9 E07 vc/mL, or about 2 E07 vc/mL. In some embodiments, the target cell density in step (a) is about 3.15 E06 vc/mL or about 5.0 E06 vc/mL.

In some embodiments of the systems and methods described herein, the production vessel has a volume between IL to 12000 L. For instance, the production vessel can have a 1 L volume, a 2 L volume, a 5 L volume, a 10 L volume, a 20 L volume, a 50 L volume, a 100 L volume, a 150 L volume, a 200 L volume, a 250 L volume, a 500 L volume, a 1000 L volume, a 1500 L volume, a 2000 L volume, a 2500 L volume, a 3000 L volume, a 3500 L volume, a 4000 L volume, a 4500 L volume, a 5000 L volume, a 5500 L volume, a 6000 L, or a 12000 L volume.

In some embodiments of the systems and methods described herein, the growth vessel and the production vessel comprise a combined growth/production vessel.

In some embodiments of the systems and methods described herein, the one or more AAV components comprise a therapeutic payload or transgene, an inverted terminal repeat (ITR) or two ITRs, one or more AAV replication and/or packaging proteins encoded by the AAV rep gene, and/or one or more AAV structural capsid proteins encoded by the AAV cap gene.

In some embodiments of the systems and methods described herein, step (b) comprises infecting the cells of step (a) with a helper virus. In some embodiments, the helper virus is an adenovirus. In some embodiments, the helper virus is Ad5.

In some embodiments of the systems and methods described herein, step (b) comprises inducing expression of one or more helper virus functions encoded by genetic material in the AAV production host cells.

In some embodiments of the systems and methods described herein, the perfusion is performed at a flow rate of about 3 mL/min/fiber to about 15 mL/min/fiber. For instance, the perfusion can be performed at a flow rate of about 3 mL/min/fiber, about 4 mL/min/fiber, about 5 mL/min/fiber, about 6 mL/min/fiber, about 7 mL/min/fiber, about 8 mL/min/fiber, about 9 mL/min/fiber, about 10 mL/min/fiber, about 11 mL/min/fiber, about 12 mL/min/fiber, about 13 mL/min/fiber, about 14 mL/min/fiber, or about 15 mL/min/fiber.

In some embodiments of the systems and methods described herein, the perfusion is performed with a medium exchange rate of about 0.5 vessel volume per day (VVD) to about 10 VVD. For instance, the perfusion can be performed with a medium exchange rate of about 0.5 VVD, about 1 VVD, about 1.5 VVD, about 2 VVD, about 2.5 VVD, about 3 VVD, about 3.5 VVD, about 4 VVD, about 4.5 VVD, about 5 VVD, about 5.5 VVD, about 6 VVD, about 6.5 VVD, about 7 VVD, about 7.5 VVD, about 8 VVD, about 8.5 VVD, about 9 VVD, about 9.5 VVD, or about 10 VVD. In some embodiments of the systems and methods described herein, the perfusion is performed with a medium exchange rate of less than 1 VVD. For instance, the perfusion can be performed with a medium exchange rate of about 0.1 VVD, about 0.15 VVD, about 0.2 VVD, about 0.25 VVD, about 0.3 VVD, about 0.35 VVD, about 0.4 VVD, about 0.45 VVD, about 0.5 VVD, about 0.55 VVD, about 0.6 VVD, about 0.65 VVD, about 0.7 VVD, about 0.75 VVD, about 0.8 VVD, about 0.85 VVD, about 0.9 VVD, or about 0.95 VVD. In some embodiments of the systems and methods described herein, the perfusion is performed at a cell-specific perfusion rate (CSPR) of about 150 pL/cell/day to about 2000 pL/cell/day, such as about 150 pL/cell/day to about 750 pL/cell/day. In some embodiments, the perfusion is performed at a CSPR of greater than 750 pL/cell/day.

In some embodiments of the systems and methods described herein, the perfusion is performed using a perfusion system selected from: Xcellerex Automated Perfusion System (APS) (Cytiva); KrosFlo® KPS TFF systems (Repligen); XCell™ ATF systems in 2, 4, 6, 8, or 10 ATF unit formats (Repligen); GEA Kytero® Single Use Pharma Separator systems (GEA); Prostak™ Microfiltration Modules systems (Millipore); Alfa Laval CultureOne™ systems (Alfa Laval); CARR® Centritech Separation Systems CARR UniFuge® Pilot or Centritech CELL 8® systems (Pneumatic Scale Angelus); Ksep® 6000S System (Sartorius); a microfluidic cell retention device; SciLog® SciPure™ systems (Parker); acoustic settler systems; and EDO Electro systems (American Piezo).

In some embodiments of the systems and methods described herein, step (a) comprises culturing the AAV production host cells in a growth medium; step (a) and step (c) comprise culturing the AAV production host cells in a growth medium; step (a) comprises culturing the AAV production host cells in a production medium; or step (a) and (c) comprise culturing the AAV production host cells in a production medium. In some embodiments of the systems and methods described herein, step (a) comprises culturing the AAV production host cells in a growth medium, and step (c) comprises culturing the AAV production host cells in a production medium. In some embodiments, production medium is perfused after infection of the AAV production host cells with the helper virus. In some embodiments, the perfusion of the production medium is initiated 1 hour-6 hours (e.g., about 1 hour-2 hours, about 2 hours-3 hours, about 3 hours-4 hours, about 4 hours-5 hours, or about 5 hours-6 hours) after infection of the cells with the helper virus.

In some embodiments of the systems and methods described herein, step (a) and step (c) comprise culturing the AAV production host cells in a blend of growth and production media. For instance, the blend of growth and production media can be a 10/90, 20/80, 30/70, 40/60, 50/50, 60/40, 70/30, 80/20, or a 90/10 blend of growth and production media.

In some embodiments of the systems and methods described herein, step (a) comprises culturing the AAV production host cells in a growth medium until about 24 hours prior to step (b), then transitioning to a production medium. The growth medium may be perfused at a rate of about 0.5 VVD to about 1 VVD until at least about 24 hours (e.g., about 96 hours, about 72 hours, about 48 hours, or about 24 hours) prior to step (b). The production medium may be perfused at a rate of about 1.5 VVD to about 2.5 VVD starting within about 24 hours prior to step (b)(e.g., 24 hours or less prior to step (b), including up to about 1 hour prior to step (b) and 0 hours prior to step (b)).

In some embodiments of the systems and methods described herein, culturing the AAV production host cells in the growth stage and/or production stage culture is carried out for about 48 hours, and/or about 2 days to about 14 days. For instance, culturing the AAV production host cells entails culturing in the production stage for a duration of about 48 hours, about 2 days, about 3 days, about 4 days, about 5 days, about 6 days, about 7 days, about 8 days, about 9 days, about 10 days, about 11 days, about 12 days, about 13 days, or about 14 days.

Perfusion may be halted after the production stage duration of about 48 hours, about 2 days, about 3 days, about 4 days, about 5 days, about 6 days, about 7 days, about 8 days, about 9 days, about 10 days, about 11 days, about 12 days, about 13 days, or about 14 days.

The systems and methods described herein may allow rAAV release from the AAV production host cells into the supernatant of the production medium. Such release may be continuous throughout the production phase. In some embodiments, the rAAV is predominantly released beginning after about 48 hours of infection of the cells with the helper virus. That is, the majority of rAAV produced may be released into the supernatant after about 48 hours of infection.

In some embodiments, the rAAV is predominantly released beginning after about 48 hours of perfusion in the production medium. That is, the majority of rAAV produced may be released into the supernatant after about 48 hours of perfusion in the production medium. In some embodiments, perfusion is stopped to retain rAAV in the supernatant. In some embodiments, perfusion is stopped at about 48 hours after infection.

Some embodiments of the systems and methods described herein further comprise harvesting the rAAV (e.g., under batch mode conditions, such as in the absence of perfusion) and/or downstream processing of the rAAV. For instance, the systems and methods may further comprise purifying, clarifying, and/or concentrating the rAAV produced according to the methods described herein. The downstream processing may comprise filtering cellular debris, colloids, or aggregates. In some embodiments, the filtering removes particles of cellular debris, colloids, or aggregates more than 1 μm in size. Some embodiments comprise purifying the rAAV using anion exchange (AEX) chromatography.

In other aspects, the present disclosure provides methods of producing a recombinant adeno-associated virus (rAAV), comprising culturing AAV production host cells using a perfusion system, wherein the AAV production host cell culture has a target cell density between 1 E06 vc/mL to 5 E07 vc/mL.

In some embodiments, the AAV production host cells are insect cells or mammalian cells. For instance, the mammalian cells may be Hela cells, Cos-7 cells, HEK293 cells, A549 cells, BHK cells, Vero cells, RD cells, or ARPE-19 cells.

In some embodiments, the AAV production host cells comprise an AAV producer cell line (PCL) comprising genetic material encoding one or more AAV components stably integrated into the AAV production host cell genome. In some embodiments, the AAV PCL may comprise one or more genetic modifications to reduce expression and/or activity of one or more genes and/or proteins to reduce production or accumulation of lactate and/or ammonia.

In some embodiments, the target cell density is about 5 E06 vc/mL to about 5 E07 vc/mL. For instance, the target cell density may be about 1 E06 vc/mL, about 2 E06 vc/mL, about 3 E06 vc/mL, about 4 E06 vc/mL, about 5 E06 vc/mL, about 6 E06 vc/mL, about 7 E06 vc/mL, about 8 E06 vc/mL, about 9 E06 vc/mL, about 1 E07 vc/mL, about 1.1 E07 vc/mL, about 1.2 E07 vc/mL, about 1.3 E07 vc/mL, about 1.4 E07 vc/mL, about 1.5 E07 vc/mL, about 1.6 E07 vc/mL, about 1.7 E07 vc/mL, about 1.8 E07 vc/mL, about 1.9 E07 vc/mL, or about 2 E07 vc/mL. In certain embodiments, the target cell density is about 3.15 E06 vc/mL or about 5.0 E06 vc/mL.

In some embodiments, the methods may be carried out in a production vessel having a volume between IL to 12000 L. For instance, the production vessel may have a IL volume, a 2 L volume, a 5 L volume, a 10 L volume, a 20 L volume, a 50 L volume, a 100 L volume, a 150 L volume, a 200 L volume, a 250 L volume, a 500 L volume, a 1000 L volume, a 1500 L volume, a 2000 L volume, a 2500 L volume, a 3000 L volume, a 3500 L volume, a 4000 L volume, a 4500 L volume, a 5000 L volume, a 5500 L volume, a 6000 L, or a 12000 L volume.

In some embodiments, the methods further comprise growing the AAV production host cells using the perfusion system to the target cell density in a combined growth/production vessel.

In some embodiments, the one or more AAV components comprise a therapeutic payload or transgene, an inverted terminal repeat (ITR) or two ITRs, one or more AAV replication and/or packaging proteins encoded by the AAV rep gene, and/or one or more AAV structural capsid proteins encoded by the AAV cap gene.

In some embodiments, the AAV production host cells are cultured at the target cell density under conditions suitable for the production of rAAV. Conditions suitable for AAV production may include infection with a helper virus to initiate AAV production such as an adenovirus (e.g., Ad5), triple transfection with AAV production plasmids, and/or induction of inducible producer cells having stable integration of components necessary to produce AAV. In some embodiments, the methods comprises initiating expression of one or more helper virus functions in the AAV production host cells thereby initiating rAAV production. In some embodiments, initiating expression of one or more helper virus functions comprises infecting the AAV production host cells with a helper virus. In some embodiments, initiating expression of one or more helper virus functions comprises inducing expression of one or more helper virus functions encoded by genetic material in the AAV production host cells. In some embodiments, the helper virus is an adenovirus. In some embodiments, the helper virus is Ad5.

In some embodiments, the perfusion system operates at a flow rate of about 3 mL/min/fiber to about 15 mL/min/fiber. For instance, the perfusion system may operate at a flow rate of about 3 mL/min/fiber, about 4 mL/min/fiber, about 5 mL/min/fiber, about 6 mL/min/fiber, about 7 mL/min/fiber, about 8 mL/min/fiber, about 9 mL/min/fiber, about 10 mL/min/fiber, about 11 mL/min/fiber, about 12 mL/min/fiber, about 13 mL/min/fiber, about 14 mL/min/fiber, or about 15 mL/min/fiber.

In some embodiments, the perfusion system operates with a medium exchange rate of about 0.5 vessel volume per day (VVD) to about 10 VVD. For instance, the perfusion system may operate with a medium exchange rate of about 0.5 VVD, about 1 VVD, about 1.5 VVD, about 2 VVD, about 2.5 VVD, about 3 VVD, about 3.5 VVD, about 4 VVD, about 4.5 VVD, about 5 VVD, about 5.5 VVD, about 6 VVD, about 6.5 VVD, about 7 VVD, about 7.5 VVD, about 8 VVD, about 8.5 VVD, about 9 VVD, about 9.5 VVD, or about 10 VVD. In some embodiments of the systems and methods described herein, the perfusion is performed with a medium exchange rate of less than 1 VVD. For instance, the perfusion can be performed with a medium exchange rate of about 0.1 VVD, about 0.15 VVD, about 0.2 VVD, about 0.25 VVD, about 0.3 VVD, about 0.35 VVD, about 0.4 VVD, about 0.45 VVD, about 0.5 VVD, about 0.55 VVD, about 0.6 VVD, about 0.65 VVD, about 0.7 VVD, about 0.75 VVD, about 0.8 VVD, about 0.85 VVD, about 0.9 VVD, or about 0.95 VVD.

In some embodiments of the systems and methods described herein, the perfusion is performed at a cell-specific perfusion rate (CSPR) of about 150 pL/cell/day to about 2000 pL/cell/day, such as about 150 pL/cell/day to about 750 pL/cell/day. In some embodiments, the perfusion is performed at a CSPR of greater than 750 pL/cell/day.

In some embodiments, the perfusion system may be one selected from: Xcellerex Automated Perfusion System (APS)(Cytiva); KrosFlo® KPS TFF systems (Repligen); XCell™ ATF systems in 2, 4, 6, 8, or 10 ATF unit formats (Repligen); GEA Kytero® Single Use Pharma Separator systems (GEA); Prostak™ Microfiltration Modules systems (Millipore); Alfa Laval CultureOne™ systems (Alfa Laval); CARR® Centritech Separation Systems CARR UniFuge® Pilot or Centritech CELL 8® systems (Pneumatic Scale Angelus); Ksep® 6000S System (Sartorius); a microfluidic cell retention device; SciLog® SciPure™ systems (Parker); acoustic settler systems; and EDO Electro systems (American Piezo).

In some embodiments, the AAV production host cells are cultured in a growth medium followed by in a production medium. The transition to the production medium may occur within about 0 hours to about 48 hours prior to infecting, or at about 24 hours prior to infecting, the AAV production host cells with helper virus. The growth medium may be perfused at a rate of about 0.5 VVD to about 1 VVD until about 24 hours prior to (e.g., 24 hours or less prior to, including up to about 1 hour prior to and 0 hours prior to) infecting the AAV production host cells with helper virus. The production medium may be perfused at a rate of about 1.5 VVD to about 2.5 VVD starting within about 24 hours prior infecting the AAV production host cells with helper virus. The AAV production host cells may be infected with a helper virus (e.g., Ad5) after the culturing in the growth medium and before the culturing in the production medium. In some embodiments, the production medium is perfused 1 hour-6 hours (e.g., about 1 hour-2 hours, about 2 hours-3 hours, about 3 hours-4 hours, about 4 hours-5 hours, or about 5 hours-6 hours) after infection of the cells with a helper virus.

In some embodiments, the AAV host production cells are cultured at the target cell density in a blend of growth and production media. For instance, the blend of growth and production media may be a 10/90, 20/80, 30/70, 40/60, 50/50, 60/40, 70/30, 80/20, or a 90/10 blend of growth and production media.

In some embodiments, the methods comprise culturing the AAV production host cells at the target cell density for about 48 hours, and/or about 2 days to about 14 days. For instance, the methods may comprise culturing the AAV production host cells at the target cell density for a duration of about 48 hours, about 2 days, about 3 days, about 4 days, about 5 days, about 6 days, about 7 days, about 8 days, about 9 days, about 10 days, about 11 days, about 12 days, about 13 days, or about 14 days.

The systems and methods described herein may allow rAAV release from the AAV production host cells into the supernatant of the production medium. Such release may be continuous throughout the production phase. In some embodiments, the rAAV is predominantly released beginning after about 48 hours of infection of the cells with a helper virus. That is, the majority of rAAV produced may be released into the supernatant after about 48 hours of infection with a helper virus.

In some embodiments, the rAAV is predominantly released beginning after about 48 hours of perfusion in the production medium. That is, the majority of rAAV produced may be released into the supernatant after about 48 hours of perfusion in the production medium. In some embodiments, perfusion is stopped to retain rAAV in the supernatant. In some embodiments, perfusion is stopped at about 48 hours. In some embodiments, perfusion is stopped at about 48 hours after infection.

In some embodiments, the methods further comprise harvesting the rAAV (e.g., under batch mode conditions, such as in the absence of perfusion) and/or downstream processing of the rAAV. For instance, the downstream processing may comprise purifying, clarifying, and/or concentrating the rAAV. The downstream processing may comprise filtering cellular debris, colloids, or aggregates. In some embodiments, the filtering removes particles of cellular debris, colloids, or aggregates more than 1 μm in size. Some embodiments comprise purifying the rAAV using anion exchange (AEX) chromatography.

These and other aspects and features of the disclosure are described in the following sections of the application.

### BRIEF DESCRIPTION OF THE DRAWINGS

The foregoing and other objects, features and advantages of the presently disclosed systems and methods will become apparent from the following description of preferred embodiments, as illustrated in the accompanying drawings. Like-referenced elements identify common features in the corresponding drawings.

FIG. 1 is a schematic of an example batch mode process (i.e., a “batch mode control process”) for AAV production.

FIG. 2 is a process flow diagram of an example process for AAV production according to the modified batch systems and methods using perfusion described herein.

FIG. 3 is a schematic of an example process for AAV production according to the modified batch systems and methods using perfusion described herein.

FIG. 4 is a bar graph representation of volumetric rAAV yield in a batch mode control process. The bar graph shows a reduction in volumetric rAAV yield with increased seeding density in a batch mode control process.

FIG. 5 is a bar graph representation of cell specific productivity in a batch mode control process. The bar graph shows a reduction in cell specific productivity with increased seeding density in a batch mode control process.

FIG. 6 is a bar graph representation of effect of pH on volumetric rAAV yield in a batch mode control process. The bar graph shows effects of pH on volumetric rAAV yield at 1.3 E06 (shown as “1.3” in the legend) and 3.15 E06 (shown as “3.15” in the legend) seeding densities in a batch mode control process.

FIG. 7 is a bar graph representation of effect of pH on cell specific productivity in a batch mode control process. The bar graph shows effects of pH on cell specific productivity at 1.3 E06 (shown as “1.3” in the legend) and 3.15 E06 (shown as “3.15” in the legend) seeding densities in a batch mode control process.

FIG. 8 is a graph showing effects of pH on accumulation of lactate at 1.3 E06 and 3.15 E06 seeding densities in a batch mode control process. (Abbreviations used in the legend: 1.3 E06 seeding density and pH 7.4 is denoted as “1.3, 7.4”; 1.3 E06 seeding density and pH 7.6 is denoted as “1.3, 7.6”; 1.3 E06 seeding density and pH 7.8 is denoted as “1.3, 7.8”; 3.15 E06 seeding density and pH 7.4 is denoted as “3.15, 7.4”; 3.15 E06 seeding density and pH 7.6 is denoted as “3.15, 7.6”; 3.15 E06 seeding density and pH 7.8 is denoted as “1.3, 7.8”).

FIG. 9A is a bar graph showing percent of viral release (AAV and helper virus) over time in a batch mode control process. FIG. 9B is a bar graph showing the rAAV release into the supernatant after infection.

FIG. 10 is a graph showing percent of viral release (AAV and helper virus) over percent of dead cells in the culture in a batch mode control process.

FIG. 11A is a bar graph representation of rAAV volumetric yield in the modified batch AAV manufacturing process and batch mode control process. The graph shows an increase in rAAV volumetric yield with increasing VVD in the modified batch AAV manufacturing process compared to batch mode control process. FIG. 11B is a bar graph representation of rAAV volumetric yield comparing low perfusion rate to a high perfusion rate process. The graph shows a further increase in rAAV volumetric yield with further increased VVD and cell density.

FIG. 12 is a bar graph representation of cell specific productivity in the modified batch AAV manufacturing process. The graph shows an increase in cell specific productivity with increasing VVD in the modified batch AAV manufacturing process.

FIG. 13 are bar graphs showing nutrient levels (glucose and glutamine, upper left and bottom left panels) as well as waste product levels (lactate and ammonium, upper right and bottom right panels) in modified batch process with medium exchange rates at 0, 1, and 2.5 VVD.

FIG. 14 is a bar graph showing volumetric rAAV yield using modified batch process at 2 L and scaled culture volumes compared to 2 L batch mode control process.

FIG. 15 is a bar graph showing cell specific productivity using modified batch process at 2 L and scaled culture volumes compared to 2 L batch mode control process.

FIG. 16 is a graph showing viable cell density (VCD) using modified batch process at 2 L and scaled culture volumes compared to 2 L batch mode control process.

FIG. 17 is a graph showing cell viability using modified batch process at 2 L and scaled culture volumes compared to 2 L batch mode control process.

FIG. 18 are graphs showing nutrient levels (glucose, left graph and glutamine, right graph) in modified batch process at 2 L and scaled culture volumes compared to 2 L batch mode control process.

FIG. 19 are graphs showing waste product levels (lactate, left graph and ammonium, right graph) in modified batch process at 2 L and scaled culture volumes compared to 2 L batch mode control process.

FIG. 20A is a graph showing percentage of full AAV capsids evaluated by Analytical Ultra Centrifugation (AUC) analysis in modified batch process at 2 L and scaled culture volumes compared to 2 L batch mode control process. FIG. 20B is a graph showing ratio of intermediate to full AAV particles with varying vessel volume per day (VVD) conditions.

FIG. 21A and FIG. 21B show use of the modified batch process with differing media compositions, capsid serotypes, and vector genomes/genes of interest. FIG. 21A are graphs showing normalized volumetric rAAV yield (left graph) and specific productivity (right graph) using batch mode control process (“Batch”) versus modified batch process (“Perf”) under varying Program, Media, and Capsid conditions (A and B). The legend in both graphs denotes rAAV titer in 1 E6 GC/mL (y axis for the left graph starts at 0.00 GC/mL to 3.50 E6 GC/mL and y axis for the right graph starts at 0.00 GC/mL to 1.40 E06 GC/mL). FIG. 21B is a graph showing harvest yield between batch mode and the modified batch process (abbreviated “HDI” in the figure) using additional Program, Media, and Capsid conditions (“Process C”).

FIG. 22 is a graph showing normalized rAAV harvest yield. The graph shows that rAAV harvest yields are reduced by the presence of lactate and/or ammonia in AAV production.

FIG. 23 is a graph showing an R² analysis of AAV cell specific yield (GC/cell) with cell-specific perfusion rates (CSPR (pL/cell/day)) up to approximately 750 pL/Cell/day.

FIG. 24A through FIG. 24C show rAAV production cost of goods (COGs) projections are reduced by the modified batch processes (e.g., intensified perfusion) described herein. FIG. 24A is a graph showing approximate projected COGs (costs of goods sold per patient dose) required per AAV yield in genome copies per milliliter (GC/mL). FIG. 24B is a graph showing approximate number of production batches required per AAV yield (GC/mL). FIG. 24C is a graph showing approximate projected COGs required per number of production batches. In FIG. 24A through FIG. 24C, diamonds or circles represents modified batch systems (e.g., intensified perfusion) described herein, while squares represent batch operating conditions.

FIG. 25 is a graph showing rAAV titer comparisons using blended media versus scheduled media transition. Solid line in the graph represents rAAV released into supernatant and dashed line in the graph represents intracellular rAAV.

FIG. 26 is a graph showing harvest titer comparisons under different scheduled media transition conditions (VVD conditions 1-4). The graph shows that harvest titer comparisons were maintained while reducing media consumption rates under different scheduled media transition conditions.

FIG. 27 is a graph representation of harvest rAAV yield for batch and HDI process. The graph compares harvest rAAV yield at 2 L in batch mode versus 2 L (denoted as MB-2 L in the figure), 50 L (denoted as MB-50 L in the figure), and 250 L (denoted as MB-250 L in the figure) using the modified batch process (abbreviated “HDI” in the figure title).

FIG. 28 is a graph representation of cell specific rAAV productivity for the batch and HDI process. The graph compares cell specific rAAV productivity at 2 L in batch mode versus 2 L (denoted as MB-2 L in the figure), 50 L (denoted as MB-50 L in the figure), and 250 L (denoted as MB-250 L in the figure) using the modified batch process (abbreviated “HDI” in the figure title).

### DETAILED DESCRIPTION OF THE INVENTION

Despite recent advances in improving productivity and scalability of AAV manufacturing processes, there remains significant need to improve existing AAV manufacturing platforms to achieve robust, high-yielding, scalable and cost-efficient processes. AAV production in suspension cell culture faces various challenges to efficient clinical and commercial scale implementation. For instance, maintaining high cell-specific productivity of viral vector product, and especially in high cell density batch culture, has proven evasive. The present systems and methods address these challenges using perfusion in a modified batch process (e.g., intensified perfusion) to improve AAV production efficiency and reduce overall cost of goods.

The present disclosure is directed to perfusion-based AAV production systems and methods that enable high volumetric productivity, reducing the high cost of goods typically associated with AAV platforms that operate in batch mode: the systems and methods of the disclosure are referred to throughout the description as “modified batch” systems and methods. In certain embodiments, the modified batch system is an intensified perfusion system and used for AAV production. Improvements to existing platform processes are described herein whereby process intensification is demonstrated to achieve a several fold increase in overall volumetric AAV yield. Without wishing to be bound by theory, the modified batch (e.g., intensified perfusion) process described herein ensures nutrient availability and cell culture waste byproduct clearance sufficient to maintain high cell density and enables multiple fold increases in AAV productivity, for example to greater than or equal to 3 E11 viral genome copies per milliliter (GC/mL)(greater than or equal to 3 E14 GC/L). These improvements, along with optimized downstream processes, can deliver a high purity AAV product suitable for human therapeutic use.

Those skilled in the art of AAV manufacture will understand that various aspects of the systems and methods described herein can be altered without deviating from the inventive subject matter of the disclosure. Aspects that can be altered or varied according to the knowledge possessed by those skilled in the art without departing from the inventions presently disclosed include, but are not limited to: AAV production scale (e.g., AAV production volume in liters), perfusion rates, AAV production cell density, growth media, production media, production feeds, media ratios, cell types, infection and/or transfection parameters or reagents, operating parameters such as pH, temperature, dissolved oxygen (DO), and the like, timing of process steps, duration of process steps, and media exchange and/or perfusion methodology (e.g., ATF, Wave Lilypad perfusion, centrifugation methods, TFF, KTF, Gea Kytero, Krosflow KPS System, and the like), among others.

### I. Definitions

Unless defined otherwise, all technical and scientific terms used herein have the same meaning as commonly understood by one of ordinary skill in the art to which this disclosure is related. Unless otherwise noted, technical terms are used according to conventional usage according to persons skilled in the art. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

It is to be noted, unless otherwise clear from the context, that the term “a” or “an” entity refers to one or more of that entity: for example, “an amino acid,” is understood to represent one or more amino acids. As such, the terms “a” (or “an”), “one or more,” and “at least one” can be used interchangeably herein.

Furthermore, the term “and/or” is to be taken as specific disclosure of each of the two or more specified features or components with or without the other(s). Thus, the term “and/or” as used in a phrase such as “A and/or B” herein is intended to include “A and B.” “A or B.” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

In order to facilitate review of the various embodiments of the disclosure, the following explanations of specific terms as used throughout this disclosure are provided below.

AAV titer, and the like: The term “AAV titer” as used herein refers to the number of viral genome copies per milliliter (GC/mL) or the viral genome copies per cell (GC/cell). In certain embodiments, the GC/mL can be determined by standard methods, including but not limited to direct quantitative PCR (qPCR) of purified vector particles, fluorescence activated cell sorting (FACS), silver stain, etc. In certain embodiments, the GC/cell can be determined by standard methods, e.g., including but not limited to dot blot, qPCR or droplet digital PCR (ddPCR), spectroscopy, or fluorimetry.

About: As used herein, the term “about” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Helper virus functions, and the like: The term “helper virus functions” as used herein encompasses genes, components, or functions, typically provided in trans, that enable production of AAV in a host cell. Helper virus functions can be provided, for example, by infection of a helper virus such as a herpes virus or adenovirus, e.g., adenovirus serotype 5 (Ad5) virus (e.g., wildtype or recombinantly engineered Ad5 helper virus). Helper virus functions can alternatively be provided in cis, for example, by incorporation of genetic material encoding helper virus functions in the host cell. Helper virus genes/functions include but are not limited to Ela, Elb, E2a, E40rf6, and VA RNA genes/functions.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are at least 13 recognized serotypes of AAV (AAV1-AAV13).

Administration/administer: To provide or otherwise expose a subject or targeted tissues of a subject with an agent, such as a therapeutic agent (e.g., a recombinant AAV), by any effective route. Exemplary routes of administration include, but are not limited to, injection (such as subcutaneous, intramuscular, intradermal, intraperitoneal, intrathecal, and intravenous), oral, intraductal, sublingual, rectal, transdermal, intranasal, vaginal and inhalation routes.

Amino acid: The term “amino acid” as used herein refers any of the twenty standard amino acids, i.e., glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., any dipeptides such as alanine-glutamine dipeptide, 4-hydroxyproline, e-V V A-tri methyllysine, 3-methylhistidine, 5-hydroxylysine, O-phosphoserine, g-carboxyglutamate, e-V-acetyllysine, co-V-methylarginine, A-acetylserine, WA′-trimethylalanine, A-formylmethionine, g-aminobutyric acid, histamine, dopamine, thyroxine, citrulline, ornithine, b-cyanoalanine, homocysteine, azaserine, and S-adenosylmethionine. In some embodiments, the amino acid is glutamate, glutamine, lysine, tyrosine, or valine. In some embodiments, the amino acid is glutamate or glutamine.

Batch culture: The term “batch culture” as used herein refers to a method of culturing cells in which all the components that will ultimately be used in culturing the cells, including the medium as well as the cells themselves, are provided at the beginning of the culturing process. A batch culture is typically stopped at some point and the cells and/or components in the medium are harvested and optionally purified.

Bioreactor: The term “bioreactor” or “culture vessel” as used herein refers to any vessel used for the growth of a cell culture (e.g., mammalian cell culture). The bioreactor can be of any size so long as it is useful for the culturing of cells (e.g., mammalian cells).

Cell culture: The terms “culture”, “cell culture”, and “eukaryotic cell culture” as used herein refer to a eukaryotic cell population, either surface-attached or in suspension that is maintained or grown in a medium under conditions suitable for survival and/or growth of the cell population. As will be clear to those of ordinary skill in the art, these terms as used herein can refer to the combination comprising the eukaryotic cell population and the medium in which the population is suspended, maintained, and/or grown.

Cell density: The term “cell density.” as used herein refers to the number of cells present in a given volume of medium. The term “target cell density” as used herein refers to the number of cells present in a volume of medium achieved during the growth stage of the culture, and used in the production stage for the production of rAAV. The term “AAV production host cell(s)” as used herein refers to the cells used for the production of rAAV.

Cell viability: The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

Coding Sequence: A “coding sequence” means the nucleotide sequence encoding a polypeptide in vitro or in vivo when operably linked to appropriate regulatory sequences. The coding sequence may or may not include regions preceding and following the coding region, e.g., 5′ untranslated (5′ UTR) and 3′ untranslated (3′ UTR) sequences, as well as intervening sequences (introns) between individual coding segments (exons).

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in cells (e.g., mammalian) or in a particular species (such as human cells). Codon optimization does not alter the amino acid sequence of the encoded protein.

Encode: The term “encode” and similar terms including “encoding” and “coding” as used herein refer to the capacity for nucleic acid molecules to carry genetic information, i.e., protein coding information, and/or biological functions. Thus, a nucleic acid that encodes a therapeutic payload may contain a genetic sequence capable of being translated into a peptide therapeutic and/or may carry information regulating the expression (transcription or translation) of a peptide therapeutic and/or give rise to a nucleic acid that has a biological function apart from encoding a peptide such as non-coding RNAs, antisense oligonucleotides (ASOs), small interfering RNAs (siRNAs), micro RNAs (miRNAs), aptamers, long non-coding RNAs (lncRNAs), and any other species of non-protein coding nucleic acid with a biological activity.

Enhancer: A nucleic acid sequence that increases the rate of transcription by increasing the activity of a promoter.

Fed batch culture: The term “fed-batch culture” as used herein refers to a method of culturing cells in which additional components are provided to the culture at some time after the beginning of the culture process. A fed-batch culture can be started using a basal medium. The culture medium with which additional components are provided to the culture at some time after the beginning of the culture process is a feed medium. The provided components typically comprise nutritional supplements for the cells which have been depleted during the culturing process. A fed-batch culture is typically stopped at some point and the cells and/or components in the medium are harvested and optionally purified.

Growth phase: “Growth phase” or “growth stage” of the cell culture refers to the period of exponential cell growth (the log phase) where cells are generally rapidly dividing. During this phase, cells are cultured for a period of time, e.g., up to 14 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and cell division. In some embodiments, during the growth phase, cells are cultured in nutrient medium containing the necessary additives generally at about 25° C.-40° C., in a humidified, controlled atmosphere, such that optimal growth is achieved for the particular cell line. In certain embodiments, cells are maintained in the growth phase for a period of about between one day and seven days, e.g., between two days to six days, e.g., six days. The length of the growth phase for the particular cells can be determined without undue experimentation. For example, the length of the growth phase will be the period of time sufficient to allow the particular cells to reproduce to a viable cell density (“vcd”) within a range of about 20%-80% of the maximal possible vcd if the culture was maintained under the growth conditions. In some embodiments, “maximum growth rate” refers to the growth rate of the specific cell line/clone measured during its exponential growth phase, while the cells are in fresh culture medium (e.g., measured at a time during culture when nutrients are sufficient and there is not any significant inhibition of growth from any components of the culture).

Intron: A stretch of DNA within a gene that does not contain coding information for a protein. RNA transcripts are processed by cellular machinery to remove introns to generate a mature, processed messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus, or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Media, medium: The terms “media”, “medium”, “cell culture medium”, “culture medium”, “tissue culture medium”, “tissue culture media”, and “growth medium” as used herein refer to a solution containing nutrients which nourish growing cultured eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no animal-derived components, hydrolysates, or components of unknown composition, and only contains components of known chemical structure or characterization. Chemically defined media are free of animal-derived components and all components have a known chemical structure. One of skill in the art understands a chemically defined medium can comprise recombinant glycoproteins or proteins, for example, but not limited to, hormones, cytokines, interleukins and other signaling molecules.

N stage, and the like: The terms “N stage,” “N culture vessel,” “second culture vessel,” “production vessel,” “production culture vessel,” “N vessel,” “N bioreactor,” “second bioreactor,” or “production bioreactor” as used herein refer to the bioreactor after the N−1 bioreactor and is used in the production of the AAV. This may alternatively be referred to throughout the present description as the “production stage” culture. As used herein, AAV producing cells in “N stage” culture are actively producing AAV product.

N-stage, and the like: The terms “N-” and “N-stage” as used herein refer to any AAV producing cell culture stage prior to initiation of N stage production.

N−1 stage, and the like: The terms “N−1 stage,” “first culture vessel,” “N−1 culture vessel,” “N−1 seed-train culture vessel,” “N−1 vessel,” “first bioreactor,” “N−1 bioreactor,” “growth vessel,” or “N−1 seed-train bioreactor” as used herein refer to a culture vessel that is immediately before the N stage culture vessel (production culture vessel) and is used to grow the cell culture to a high viable cell density for subsequent inoculation into N (production stage) culture vessel. The cell culture to be grown in the N−1 culture vessel may be obtained after culturing the cells in several vessels prior to the N−1 culture vessel, such as N−4, N-3, and N-2 vessels. The N−1 stage may alternatively be referred to throughout the present description as the “growth stage” culture. As used herein, AAV producing cells in the “N−1 stage” culture may be in the growth phase.

Nutrient media, feed media, and the like: The term “nutrient media,” “feed media.” “feed,” “total feed.” and “total nutrient media” as used herein can be used interchangeably and include a “complete” media used to grow, propagate, and add biomass to a cell line. Nutrient media is distinguished from a substance or simple media which by itself is not sufficient to grow and propagate a cell line. Thus, for example, glucose or simple sugars by themselves are not nutrient media, since in the absence of other required nutrients, they would not be sufficient to grow and propagate a cell line. One of skill in the art can appreciate that cells may continue to grow, live, and propagate in the presence of incomplete media, but become unstable and/or greatly reduce their growth rate. Thus, in some embodiments, the term “nutrient media” includes a media sufficient to grow, propagate, and add biomass to a cell line without a loss in stability, growth rate, or a reduction of any other indicators of cellular health for a period of at least 2 days, 3 days, 4 days, 5 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, or 18 weeks. In some embodiments, the term “nutrient media” includes a media which may lack one or more essential nutrients, but which can continue to grow, propagate, and add biomass to a cell line without a loss in stability, growth rate, or a reduction of any other indicators of cellular health for a period of at least 2 days, 3 days, 4 days, 5 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, or 18 weeks. In some embodiments, the nutrient media is a cell culture media. Optimal cell culture media compositions vary according to the type of cell culture being propagated. In some embodiments, the nutrient media is a commercially available media. In some embodiments, the nutrient media contains e.g., inorganic salts, carbohydrates (e.g., sugars such as glucose, galactose, maltose or fructose), amino acids, vitamins (e.g., B group vitamins (e.g., B12), vitamin A, vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum, newborn calf serum and horse serum), trace elements (e.g., zinc, copper, selenium and tricarboxylic acid intermediates), hydrolysates (hydrolyzed proteins derived from plant or animal sources), and combinations thereof. Examples of nutrient medias include, but are not limited to, basal media (e.g., minimum essential medium (MEM), Dulbecco's modified eagle medium (DMEM), Glasgow minimum essential medium GMEM), complex media RPMI media (e.g., RPMI 1640), Iscoves DMEM, Leibovitz L-15, Leibovitz L-15, TC 100), serum free media (e.g., Chinese Hamster Ovary (CHO) media, Ham's F10 and derivatives, Ham F12, DMEM/F12). Common buffers found in nutrient media include phosphate buffered saline (PBS), Hank's balanced salt solution (BSS), Earle's salts (Earle's salt solution), Dulbecco's phosphate-buffered saline (DPBS), Hank's BSS (HBSS), and Earle's BSS (EBSS). Media for culturing mammalian cells are well known in the art and are available from, e.g., Sigma-Aldrich Corporation (St. Louis, MO), HyClone (Logan, UT), Invitrogen Corporation (Carlsbad, CA), Cambrex Corporation (E. Rutherford, NJ), Irvine Scientific (Santa Ana, CA), Gibco/ThermoFisher Scientific, and others. Other components found in nutrient media can include ascorbate, citrate, cysteine/cystine, glutamine, folic acid, glutathione, linoleic acid, linolenic acid, lipoic acid, oleic acid, palmitic acid, pyridoxal/pyridoxine, riboflavin, selenium, thiamine, and transferrin. One of skill in the art will recognize that there are modifications to nutrient media which would fall within the scope of the presently disclosed systems and methods.

Perfusion culture: The terms “perfusion culture,” “perfusion system,” “perfusion,” and the like as used herein refers to a method of culturing cells in which additional components are provided continuously or semi-continuously to the culture after the beginning of the culture process. The provided components typically comprise nutritional supplements for the cells which have been depleted during the culturing process. A portion of the cells and/or components in the medium are typically harvested on a continuous or semi-continuous basis and are optionally purified.

Pharmaceutically acceptable carrier: The pharmaceutically acceptable carriers (vehicles) useful in this disclosure may be those conventionally used in the art. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules, or agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions for example, powder, pill, tablet, or capsule forms), conventional nontoxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, protein: The terms “polypeptide” or “protein” as used herein refer a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. If a single polypeptide is the discrete functioning unit and does require permanent physical association with other polypeptides to form the discrete functioning unit, the terms “polypeptide” and “protein” as used herein are used interchangeably. If discrete functional unit is comprised of more than one polypeptide that physically associate with one another, the term “protein” as used herein refers to the multiple polypeptides that are physically coupled and function together as the discrete unit. The term “protein” as used herein is intended to encompass a singular “protein” as well as plural “proteins.” Thus, as used herein, terms including, but not limited to “peptide,” “polypeptide,” “amino acid chain,” or any other term used to refer to a chain or chains of amino acids, are included in the definition of a “protein,” and the term “protein” may be used instead of, or interchangeably with, any of these terms. The term further includes proteins which have undergone post-translational modifications, for example, glycosylation, acetylation, phosphorylation, amidation, derivatization by known protecting/blocking groups, proteolytic cleavage, or modification by non-naturally occurring amino acids. Proteins also include polypeptides which form multimers, e.g., dimers, trimers, etc. The term protein also includes fusions proteins, e.g., a protein that is produced via a gene fusion process in which a protein (or fragment of a protein) is attached to an antibody (or fragment of antibody).

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible. As used herein, gene-specific endogenous promoter refers to native promoter element that regulates expression of the endogenous gene of interest. In an exemplary embodiment, a therapeutic payload is a human and the promoter is a gene-specific endogenous promoter that regulates expression of the human gene in a native context.

Purified: The term “purified” does not require absolute purity: rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus, or other active compound that has been isolated from a cell, cell culture medium, or other crude preparation and subjected to fractionation (e.g., filtration) to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of one or more sequences. This artificial combination can be accomplished by chemical synthesis or by the artificial manipulation of isolated segments of nucleic acid molecules, such as by genetic engineering techniques. Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein, or virus. As used herein, “recombinant AAV” or “rAAV” may refer to an AAV particle in which a recombinant nucleic acid molecule such as a recombinant nucleic acid molecule encoding a therapeutic payload has been packaged.

Seeding: The term “seeding” as used herein refers to the process of providing a cell culture to a bioreactor or another vessel (e.g., growth vessel or production vessel). In one embodiment, the cells have been propagated previously in another bioreactor or vessel (e.g., growth vessel or production vessel). In another embodiment, the cells have been frozen and thawed immediately prior to providing them to the bioreactor or vessel (e.g., growth vessel or production vessel). The term refers to any number of cells, including a single cell.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: As used herein, the term “subject” refers to a human or any non-human animal (e.g., mouse, rat, rabbit, dog, cat, cattle, swine, sheep, horse, or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient” unless the context requires otherwise. A subject can be afflicted with or is susceptible to a disease or disorder but may or may not display symptoms of the disease or disorder.

Untranslated region (UTR): A typical mRNA contains a 5′ untranslated region (5′ UTR) and a 3′ untranslated region (3′ UTR) upstream and downstream, respectively, of the coding region (see Mignone, F., Gissi, C., Liuni, S. et al. Untranslated regions of mRNAs. Genome Biol 3, reviews0004.1 (2002)).

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector. Depending on the context, a broader definition of the term “vector” may apply, which signifies a carrier or transmitter, i.e., usually a biological carrier of a biological agent.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Overview

The present disclosure is directed to perfusion-based AAV production systems and methods that enable high volumetric productivity, reducing the high cost of goods typically associated with comparable AAV platforms (e.g., non-intensified AAV platforms). The systems and methods of the present disclosure may be used in the manufacture of any viral particle, for example an adeno-associated viral particle, e.g., for therapeutic applications. The systems and methods described herein can be incorporated into procedures implementing producer cell lines (e.g., mammalian producer cell lines) for the production of viral particles, e.g., producer cell lines in suspension, or, alternatively into procedures implementing multiple, e.g., triple, transfection approaches to give rise to viral particle-producing cells. In some embodiments, the systems and methods disclosed herein incorporate transfection master mixes according to those described, e.g., in US patent application Publication Number US20200124505A1.

Thus, provided herein are systems and methods of producing viral particles, e.g., AAV. The modified batch (e.g., intensified perfusion) systems and methods described herein mitigate challenges in the bioreactor production field of maintaining high specific productivity in batch mode and other perfusion culture systems (e.g., non-intensified), which challenges are emphasized at heightened cell densities, e.g., about 1 E06 cells/mL or greater. The systems and methods described herein achieve up to five-fold or greater increases in volumetric productivity compared to existing methods for AAV production, thereby reducing cost of goods and improving efficiency of AAV production.

This disclosure provides various systems and methods to be used for AAV manufacturing. The disclosure demonstrates how to increase AAV production through use of modified batch systems (e.g., intensified perfusion systems) and methods and optionally use of increased cell density in a growth stage vessel under conditions to enrich the culture medium and remove inhibitory metabolites during production stage. In particular, systems and methods are disclosed which enable culturing of AAV producing cells in a single vessel (e.g., bioreactor) from the growth stage through the AAV production stage, in some instances while maintaining high cell density, e.g., at or greater than 1 E06 cells/mL. The systems and methods described herein are unique from other systems, which utilize perfusion in the growth stage, prior to the production stage for gene therapy viruses, to achieve high density culture and then transfer that high cell mass to the production vessel for batch mode operation. In contrast, the presently described systems and methods enable perfusion of the growth and production stages, including—in some embodiments—in the same vessel, and allow cell culture medium-exchange after infection with helper virus or after transfection with helper virus components to enrich the culture medium and remove inhibitory waste products from the culture medium, thereby improving AAV productivity. When compared to the batch mode operation at low cell density as well as batch mode operation at higher cell density (e.g., greater than 1 E06 cells/mL), the systems and methods described herein lead to significant and multi-fold-increases in overall volumetric AAV productivity while maintaining cell specific productivity.

In batch mode AAV manufacturing processes generally, cell expansion follows producer cell line (e.g., mammalian producer cell line) thaw and expansion in shake-flasks. Expanded producer cells are transferred to intermediate scale bioreactors before being transferred to an N−1 growth vessel at, e.g., 250 L scale (e.g., 10× smaller than final production stage). Systems useful for N−1 growth stage culture are commercially available and include, for example, Repligen Xcell ATF™ 6 single use alternating tangential flow filtration system and comparable systems. Generally, these systems can achieve a cell density of >13 E06 viable cells per milliliter (vc/mL). Next, the cell mass is split approximately 1:10 from N− (e.g., N−1) in growth medium to N stage production at, e.g., 2000 L scale, at approximately 1.3 E06 vc/mL. Typically, this entails a new production stage vessel separate from the growth stage vessel and requires different production medium and feeds. After transfer to N stage or production stage culture, AAV production is initiated by helper virus, e.g., an adenovirus such as adenovirus serotype 5 (Ad5), infection in a 90:10 production medium and growth medium mixture and carried for about 4 days in production culture. Finally, harvest operations are conducted within the bioreactor, e.g., nuclease addition and pre-treatment activities, and subsequent downstream filter clarification, AAV purification and helper virus removal processes are carried out.

In the modified batch (e.g., intensified perfusion) systems and methods described herein, the cell seed-train cell mass is thawed and carried in shake-flasks in the same manner as in batch mode AAV manufacturing processes generally. The bioreactor expansion stage includes an additional vessel at, e.g., 250 L scale (without use of alternating tangential flow filtration). Importantly, in some embodiments, subsequent growth and production stages can be, but need not be, carried out in the same, e.g., 2000 L vessel (bioreactor), without the need to transfer a high cell density seed train to a new vessel for production stage culture. For the growth stage at 2000 L scale, a final high cell density target of, for example, 5 E06 vc/mL-50 E06 vc/mL is achievable using a blend of growth and production medium. Once the desired cell mass reaches the high cell density target (e.g., 5 E06 vc/mL or higher), the perfusion-mediated or other media exchange may be halted. AAV production is then initiated by helper virus, e.g., adenovirus, infection or transfection with helper virus components and feed addition after about 1 hour to about 2 hours, and perfusion-mediated or other media exchange is initiated again thereafter with appropriate production medium and feeds. In some embodiments of the systems and methods described herein, alternating tangential flow (ATF) perfusion is utilized in production stage culture. ATF operation may remain active for, e.g., about 1 day to about 2 days post-infection with helper virus, e.g., adenovirus, or transfection with helper virus components to enrich the medium and eliminate accumulation of waste products. After the about 1 day to about 2 days, perfusion-mediated or other media exchange is terminated to allow accumulation of AAV particles in culture supernatant. In some embodiments, ATF operation may remain active for longer than 2 days post-infection with helper virus or transfection with helper virus components, e.g., up to about 14 days induction of AAV production. Release of AAV particles into the supernatant occurs, e.g., after about 12 hours, after about 24 hours, after about 36 hours, after about 48 hours, after about 60 hours, after about 72 hours, after about 84 hours, or after about 96 hours, depending, e.g., on the producer cell line being used. After a total of about 4 days to about 6 days post-infection with helper virus or transfection with helper virus components, AAV harvest procedures are performed for subsequent downstream process purification such as filter clarification, AAV purification and helper virus, e.g., adenovirus such as Ad5, removal. In some embodiments, helper virus removal processes include heat inactivation processes such as those described in, e.g., US patent application Publication Number US20190083554A1. In some embodiments, tangential flow depth filtration can be implemented to integrate AAV harvest and clarification processes into a single step (see Mendes, João P., et al. “AAV process intensification by perfusion bioreaction and integrated clarification.” Frontiers in Bioengineering and Biotechnology 10 (2022)).

In addition to employing the modified batch systems (e.g., intensified perfusion) and methods using perfusion described herein, further approaches to increasing AAV production and/or AAV titer can be implemented in the systems and methods disclosed. For example, the systems and methods disclosed can be further refined to include: use of glucocorticoid analogs as described, e.g., in US patent application Publication Number US20190290710A1; use of a compound selected from the group consisting of niacinamide, niacin, methyl-nicotinate, nicotinyl alcohol, and any combination(s) thereof as described, e.g., in PCT Publication Number WO/2021/188449; optimized downstream clarification processes as described, e.g., in US patent Application Publication Number US20200048641A1; use of tonicifying agents as described in, e.g., US patent application Publication Number US20210277416; and/or co-delivery of Rep mRNA or HDAC inhibitors as described, e.g., in US patent application Publication Number US20200032221A1.

In some embodiments, the modified batch systems and methods using perfusion (e.g., intensified perfusion) described herein increase infection density about 2-fold, about 3-fold, about 4-fold, about 5-fold, or greater than 5-fold from optimal batch mode or other conditions (e.g., non-intensified), resulting in increased AAV productivity to greater than or equal to, for example, 3 E11 genome copies per milliliter (GC/mL), i.e., greater than or equal to 3 E14 GC/L.

The presently described systems and methods are distinct from prior methods demonstrating perfusion in the growth stage to achieve high density culture for transfer to the production vessel for batch mode operation. While others have demonstrated perfusion capability during AAV production stage in transient transfection systems and in HEK based cell systems during AAV production, none have demonstrated the use of perfusion during AAV production using, e.g., HeLa producer cells, e.g., HeLa producer cells leveraging infection with a helper virus such as adenovirus. Some embodiments of the systems and methods disclosed herein enable high cell density production utilizing perfusion equipment to combine the growth stage and production stage production into a single step in the same vessel, thereby increasing overall volumetric productivity. In some embodiments, the use of perfusion technology enables high producer cell density (e.g., HeLa PCL density) in the production vessel. Subsequently, a change to production medium through medium-exchange after infection with helper virus enriches the culture medium and removes inhibitory waste products, leading to increased AAV productivity. When compared to the batch mode operation, e.g., at lower cell density, as well as batch mode operation at higher density (i.e., greater than about 1 E06 cells/mL), the act of perfusion after initiation of AAV production leads to significant fold-increase in overall volumetric AAV productivity and maintains cell specific productivity similar to that seen in batch mode operation.

The systems, methods, reagents and components and features thereof will now be described in more detail in the following sections.

### III. Viral Vectors

This section describes viral vectors generally, including AAV vectors, which can be produced by the modified batch systems and methods using perfusion (e.g., intensified perfusion) described herein. In some aspects, the present disclosure provides systems and methods for production of adeno-associated virus (AAV). In some embodiments, the present disclosure provides systems and methods for production of recombinant adeno-associated virus (rAAV) comprising an adeno-associated virus (AAV) capsid, and a recombinant vector genome packaged therein.

The AAV, e.g., rAAV, to be produced may contain a packaged vector genome that comprises, e.g., an AAV 5′-ITR, a promoter sequence, a partial or complete coding sequence for a therapeutic payload, and an AAV 3′-ITR. In some embodiments, the packaged vector genome may further comprise an enhancer sequence, an intron sequence, a consensus Kozak sequence, and/or a polyadenylation signal sequence. In some embodiments, the packaged vector genome can further include one or more stuffer nucleic acid sequences. In some embodiments, a stuffer nucleic acid sequence is situated between the intron and the partial or complete coding sequence for the therapeutic payload.

In some embodiments, the AAV vector is a recombinant AAV vector (rAAV). For brevity, the term “AAV vector” and the like as used herein comprises the sub-genus rAAV, such that mention of an AAV vector encompasses AAV vectors and rAAVs. The AAV vectors produced by the systems and methods of the present disclosure can comprise a capsid of any AAV serotype. For example, the disclosed systems and methods can be used to produce AAV comprising a capsid of serotype 1, 2, 3, 4, 5, 6, 7, 8, 9, 10, 11, 12, 13, rh10, hu37 (i.e., AAV1, AAV2, AAV3, AAV4, AAV5, AAV6, AAV7, AAV8, AAV9, AAV10, AAV11, AAV12, AAV13, AAVrh10, AAVhu37), as well as any one of the more than 100 variants isolated from human and nonhuman primate tissues. See, e.g., Choi et al., 2005, Curr Gene Ther. 5:299-310, 2005: Gao et al., 2005, Curr Gene Ther. 5:285-297; and Drouin et al. Future Virol. 2013 December; 8(12):1183-1199. Beyond the aforementioned capsids, the disclosed systems and methods can be used to produce AAV comprising variant AAV capsids which have been engineered to harbor one or more beneficial therapeutic properties (e.g., improved targeting for select tissues, increased ability to evade the immune response, reduced stimulation of neutralizing antibodies, etc.). Non-limiting examples of such engineered variant capsids are described in U.S. Pat. Nos. 9,506,083, 9,585,971, 9,587,282, 9,611,302, 9,725,485, 9,856,539, 9,909,142, 9,920,097, 10,011,640, 10,081,659, 10,179,176, 10,202,657, 10,214,566, 10,214,785, 10,266,845, 10,294,281, 10,301,648, 10,385,320, and 10,392,632 and in PCT Publication Nos. WO/2017/165859, WO/2018/022905, WO/2018/156654, WO/2018/222503, and WO/2018/226602. These and other AAV serotype variants may likewise be produced by the systems and methods of AAV production herein. The selection of AAV serotype and/or variant to be produced will depend in part on the cell type(s) to be targeted by the AAV vector, for example, in a gene therapy application.

### A. Vector Genome Components

In some embodiments, the AAV vectors produced by the systems and methods described herein comprise a vector genome packaged within the AAV vector.

In some embodiments, the AAV vectors produced by the systems and methods described herein include a vector genome comprising an AAV ITR sequence which functions as both the origin of vector DNA replication and the packaging signal of the vector genome, i.e., when AAV and adenovirus helper virus functions are provided in trans. Additionally, the ITRs may serve as the target for single-stranded endonucleatic nicking by the large Rep proteins, resolving individual genomes from replication intermediates.

In some embodiments, the AAV vectors produced by the systems and methods described herein include a vector genome comprising a promoter sequence which helps drive and regulate transgene expression, e.g., expression of the therapeutic payload. The promoter sequence can be (or be derived from) a ubiquitous promoter or a tissue-specific promoter sequence. In some embodiments, the promoter sequence can be a chicken beta (β)-actin (CBA) promoter sequence, a cytomegalovirus (CMV) immediate early gene promoter sequence, a transthyretin (TTR) promoter sequence, a thyroxine binding globulin (TBG) promoter sequence, an alpha-1 antitrypsin (A1AT) promoter sequence, a CMV early enhancer/chicken β actin (CAG) promoter sequence, or therapeutic payload gene-specific endogenous promoter sequence. In some embodiments, the promoter sequence is located downstream of an enhancer sequence. In some embodiments, the promoter sequence is located upstream of an intron sequence. In some embodiments, the promoter sequence is located between the selected 5′-ITR sequence and the therapeutic payload coding sequence, including any 5′-untranslated region (UTR).

In addition to a promoter, a packaged genome may contain other appropriate transcription initiation, termination, enhancer sequence, and efficient RNA processing signals. As described in further detail below, such sequences include splicing and polyadenylation (poly A) signals, regulatory elements that enhance expression (i.e., WPRE), sequences that stabilize cytoplasmic mRNA, sequences that enhance translation efficiency (i.e., the Kozak consensus sequence), and sequences that enhance protein stability.

In some embodiments, the packaged vector genome further comprises a consensus Kozak sequence. In some embodiments, the consensus Kozak sequence is located downstream of an intron sequence. In one embodiment, the consensus Kozak sequence is GCCGCC. As will be understood by those skilled in the art, the consensus Kozak sequence is typically located immediately upstream of a coding sequence such as a sequence coding for a therapeutic payload. As will be appreciated by the skilled artisan, the consensus Kozak sequence can be considered to share an ATG residue corresponding to the start codon of the therapeutic payload polypeptide.

In some embodiments, the packaged vector genome further comprises a 5′-untranslated region (UTR). The 5′ UTR can be from an endogenous gene-specific mRNA with desirable expression characteristics. 5′ UTRs have been known to play an important role in optimizing transgene production by competing with cellular transcripts for translation initiation factors and ribosomes, increasing mRNA half-life by minimizing mRNA decay or post-transcriptional gene silencing, and avoiding deleterious interactions with regulatory proteins or inhibitory RNA secondary structures (see Chiba, Y., and Green, P. (2009). J. Plant Biol. 52, 114-124, Moore, M. J., and Proudfoot, N. J. (2009). Cell 136, 688-700, and Jackson, R. J., et. al. (2010). Nat. Rev. Mol. Cell Biol. 11, 113-127).

Similarly, the packaged vector genome may further comprise a 3′ UTR. The 3′ UTR may be situated downstream of the coding sequence for the therapeutic payload. 3′ UTRs have been shown to be involved in numerous regulatory processes including transcript cleavage, stability and polyadenylation, translation and mRNA localization (see Barrett, L. W., et. al. (2012). Cell Mol Life Sci. November: 69 (21): 3613-3634).

In some embodiments, the coding sequence for a therapeutic payload is a partial or complete coding sequence for a therapeutic polypeptide or polynucleotide. Exemplary therapeutic polypeptides can be found in PCT Publication No. WO/2021/067598 and include, e.g., polypeptides that may be useful in the treatment of mammals, e.g., humans, including ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit a (PCCA) and propionyl-CoA carboxylase subunit b (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin). A non-limiting list of therapeutic payloads may also be found in PCT Publication No. WO/2019/168961. The therapeutic payload may be a wild-type coding sequence for a protein or polypeptide, a codon-optimized coding sequence for a protein or polypeptide, or a sequence encoding a functional nucleotide species such as a non-coding RNA, or a combination thereof. Examples of functional nucleotide species suitable as therapeutic payloads include non-coding RNAs, antisense oligonucleotides (ASOs), small interfering RNAs (siRNAs), micro RNAs (miRNAs), aptamers, long non-coding RNAs (lncRNAs), and any other species of non-protein coding nucleic acid with a biological activity relevant to a disease condition. For example, the therapeutic payload may be a morpholino, a peptide-linked morpholino, an antisense oligonucleotide (ASO), a phosphorodiamidate morpholino oligomer (PMO), a therapeutic transgene, a polynucleotide encoding a therapeutic polypeptide or peptide, a peptide-conjugated PMO, one or more peptides, one or more polynucleotides encoding a CRISPR-Cas protein, a guide RNA, or both a CRISPR-Cas protein and a guide RNA, a ribonucleoprotein comprising a CRISPR-Cas system molecule, a therapeutic transgene RNA, or other gene-modifying or therapeutic RNA and/or protein, or any combination thereof. As used herein, the term “wild-type” refers to a biopolymer (e.g., a polypeptide sequence or polynucleotide sequence) that is the same as a biopolymer (e.g., polypeptide sequence or polynucleotide sequence) that exists in nature.

In some embodiments, the packaged vector genome further comprises one or more enhancer sequences. In some embodiments, the enhancer sequence is (or is derived from) a cytomegalovirus immediate early gene (CMV) enhancer sequence, a transthyretin enhancer (enTTR) sequence, a chicken beta (β)-actin (CBA) enhancer sequence, an En34 enhancer sequence, or an apolipoprotein E (ApoE) enhancer sequence.

In some embodiments, the packaged vector genome further comprises one or more intron sequences. The intron sequence may be (or may be derived from) an SV40 Small T intron sequence, a rabbit hemoglobin subunit beta (rHBB) intron sequence, a human beta globin IVS2 intron sequence, a Promega chimeric intron sequence (β-globin/IgG chimeric intron sequence), or an hFIX intron sequence.

In some embodiments, the packaged vector genome further comprises a polyadenylation signal sequence. In some embodiments, the polyadenylation signal sequence is (or is derived from) a bovine growth hormone (BGH) polyadenylation signal sequence, an SV40 polyadenylation signal sequence, a rabbit beta globin polyadenylation signal sequence, a gene-specific endogenous polyadenylation signal sequence.

### B. AAV Capsids

The present disclosure provides systems and methods for producing AAV particles comprising an AAV capsid of any AAV serotype. In some embodiments, the AAV capsid is from an AAV of serotype 1, 2, 3, 4, 5, 6, 7, 8, 9, 10, 11, 12, 13, rh10, or hu37 (i.e., AAV1, AAV2, AAV3, AAV4, AAV5, AAV6, AAV7, AAV9, AAV10, AAV11, AAV12, AAV13, AAVrh10, or AAVhu37). In an exemplary embodiment, the AAV capsid is from an AAV serotype 9 (AAV9) vector, an AAV9 variant vector, an AAV serotype 8 (AAV8) vector, an AAV serotype 2 (AAV2) vector, or any other known AAV serotype vector or variant. International Patent Application Publication No. WO2021041485A1 at paragraphs [0115]-[0131], provides a listing of known AAV serotypes, variants, mutations, and derivatives thereof, any of which may be suitable for AAV production using the systems and methods described herein.

### C. Pharmaceutical Compositions

The systems and methods for AAV production provided herein can be used to generate pharmaceutical compositions comprising AAV vectors. Pharmaceutical compositions comprising the AAV vectors produced according to the methods disclosed herein may further comprise a pharmaceutically acceptable carrier. Pharmaceutical compositions comprising the AAV vectors produced according to the methods disclosed herein may further be formulated for administration to a subject. Suitable pharmaceutical compositions formulated for administration of AAV can be found, for example, in U.S. Patent Application Publication No. 2012/0219528. The pharmaceutically acceptable carriers (vehicles) useful in AAV compositions are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents, including, e.g., AAV produced according the systems and methods of the present disclosure.

The present disclosure relates in some aspects to pharmaceutical compositions comprising an AAV produced according to the presently disclosed methods. In some embodiments, the pharmaceutical composition comprises a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In some embodiments, the AAV is formulated in a buffer/carrier suitable for infusion in human subjects. The buffer/carrier may include a component that prevents the AAV from sticking to the infusion tubing but does not interfere with the AAV binding activity in vivo. Various suitable solutions may include one or more of: a buffering saline, a surfactant, and a physiologically compatible salt or mixture of salts adjusted to an ionic strength equivalent to, e.g., about 100 mM sodium chloride (NaCl) to about 250 mM sodium chloride, or a physiologically compatible salt adjusted to an equivalent ionic concentration. The pH of the formulated pharmaceutical composition may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene 10 (poly(propylene oxide)) flanked by two hydrophilic chains of polyoxyethylene (polyethylene oxide)), SOLUTOL HS 15 (Macrogol-15 hydroxystearate), LABRASOL (polyoxy caprylic glyceride), polyoxy 10 oleyl ether, TWEEN (polyoxyethylene sorbitan fatty acid esters), ethanol and/or polyethylene glycol.

### IV. Methods of Producing AAV

The present disclosure provides systems and methods for production of adeno-associated viruses (AAV), which can be used as therapeutic payload (e.g., gene) delivery vehicles. In some embodiments, the AAV produced by the systems and methods provided herein comprise a wild-type or engineered (mutant or variant) AAV capsid packaged with an engineered vector genome comprising, e.g., a transgene, e.g., a transgene encoding a therapeutic payload, e.g., a therapeutic protein. In some embodiments, such engineered AAV can be administered to a subject in need of the therapeutic payload, and upon administration, the therapeutic payload can be expressed in the subject's own cells. The systems and methods described herein are applicable to an AAV comprising any therapeutic payloads.

### A. AAV Production Host Cells

The systems and methods provided herein utilize host cells for production of AAV vectors including rAAV. In some embodiments, the host cells comprise a recombinant nucleic acid molecule, viral vector, e.g., an AAV vector, or a rAAV disclosed herein. In specific embodiments, the host cells are suitable for propagation of AAV. In specific embodiments, the host cells are suitable for production of AAV. In specific embodiments, the host cells are suitable for propagation and/or production of AAV.

Any known AAV production host cell can be used. Examples of known host cell types include bacteria cells, yeast cells, insect cells (such as Sf9 cells), and mammalian cells, etc. In some embodiments, the host cell can be a cell (or a cell line) appropriate for production of AAV (e.g., rAAV), for example, a HeLa cell, Cos-7 cell, HEK293 cell (and HEK293 derivative cell lines), A549 cell, BHK cell, Vero cell, RD cell, or ARPE-19 cell. In some embodiments, stable inducible AAV producer cell lines, such as those according to the doxycycline-inducible CAP cells and HEK293 cells described in WO 2022/112218 A1 are utilized in the systems and methods described herein. Additional host cell types are described elsewhere herein.

In some embodiments, recombinant nucleic acid molecules or vectors can be delivered into the host cell culture using any suitable method known in the art (e.g., transfection-based methods). In some embodiments, a stable host cell line that has a recombinant nucleic acid molecule or vector inserted into its genome by transfection is utilized in the systems and methods provided herein. After transfection of a AAV vector to the host cell, integration of the AAV vector into the host cell genome can be assayed by various methods, such as antibiotic selection, fluorescence-activated cell sorting, southern blot, PCR based detection, fluorescence in situ hybridization as described by Nakai et al, Nature Genetics (2003) 34, 297-302: Philpott et al, Journal of Virology (2002) 76(11): 5411-5421, and Howden et al, J Gene Med 2008:10:42-50. Furthermore, a stable cell line can be established and utilized according to protocols known in the art, such as those described in Clark, Kidney International Vol 61 (2002):S9-S15, and Yuan et al, Human Gene Therapy 2011 May; 22(5): 613-24.

In some embodiments, the host cells utilized for AAV production are engineered to be AAV producer cells. An AAV producer cell line (PCL) thus may be utilized in the systems and methods described herein.

Various aspects of culturing AAV PCLs in systems and methods of the present disclosure are described here. For example, parameters of PCL culture conditions, including media exchange technology, perfusion, perfusion rates, cell density, media, media supplementation, cell viability, growth rate, pH, culture duration, culture volume, and seeding density, among others, which may be selected or utilized in the presently disclosed systems and methods, are described.

Various cellular/viral components are needed for AAV production. For example, components for AAV replication, vector genome packaging, and structural components of the capsid all must be produced to generate an AAV particle. The AAV Rep gene encodes four proteins that are involved in packaging and replication, and the cap gene encodes three structural capsid proteins (called VP1, VP2, and VP3). Wild-type AAV is replication deficient and requires co-infection of cells by a helper virus, e.g., a herpes virus or adenovirus, e.g., Ad5 virus, in order to replicate. For example, Ad5 virus supplies Ad5 helper virus function factors/genes, such as Ela, Elb, E40rf6, E2a and/or virus-associated (VA) RNA, that mediate AAV replication. See, e.g., Nayak et al. J Virol. 81.5 (2007): 2205-12. Recombinant AAV vectors permit integration of a gene of interest, or transgene, into a viral vector such that the transgene is transmitted, encoded, and/or expressed by the viral machinery. In some cases, a recombinant AAV vector comprises inverted terminal repeats (ITRs) that serve as origins of replication and/or packaging. In some embodiments, the recombinant AAV vector comprises a transgene flanked by ITRs (one ITR on either side of the transgene). See, e.g., Carter B. Adeno-Associated Virus and AA V Vectors for Gene Delivery, in Gene and Cell Therapy, 4^{th} Edition, N. S. Templeton, Editor. 2015, CRC Press.

AAV PCLs can be generated that contain one or more of these components required for AAV production. An exemplary list of AAV components includes (a) a nucleic acid sequence comprising a transgene, e.g., a therapeutic payload: (b) a nucleic acid sequence comprising an inverted terminal repeat (ITR), e.g., one or two ITRs, e.g., where the two ITRs flank one or more additional AAV components (e.g., a transgene); (c) a nucleic acid sequence encoding one or more AAV replication and/or packaging proteins (e.g., encoded by the AAV rep gene); (d) a nucleic acid sequence encoding one or more AAV structural capsid proteins (e.g., encoded by the AAV cap gene, e.g., VP1, VP2, or VP3 protein); (e) one or more AAV replication and/or packaging proteins: (f) one or more AAV structural capsid proteins; and/or (g) one or more helper virus components, e.g., Ad5 helper virus components (e.g., E1a, E1b, E40rf6, E2a and/or VA RNA: or Ad5 helper virus). As used herein, an “AAV PCL” or “AAV producer cell line” refers to a cell, e.g., a cell described herein, that comprises one or more of (a)-(g) above and, when supplied with any necessary helper virus component(s), is capable of producing an AAV under suitable production conditions that are well known in the art.

In some embodiments, any combination of the AAV components (e.g., (a)-(d) or (g) above) are provided on the same nucleic acid molecule or on separate nucleic acid molecules (e.g., on one, two, three, or four or more separate nucleic acid molecules). In some embodiments, the transgene is flanked on both sides by an ITR.

In some embodiments, a PCL comprises any combination of (a)-(g) above. In some embodiments, the PCL can comprise recombinant AAV vector(s) and/or Rep and/or cap genes, e.g., stably integrated into a permissive host cell. In some embodiments, the PCL comprises a nucleic acid sequence encoding a transgene and a nucleic acid sequence comprising one or more ITRs, e.g., where the transgene is flanked on either side by one ITR. In other embodiments, a PCL comprises a packaging cell line that comprises rep and/or cap genes but not the vector/transgene (and the vector/transgene can be supplied by a separate virus, e.g., recombinant adenovirus, e.g., Ad5). PCLs comprising other combinations of AAV components can be made. Any such PCLs and other variations of PCLs can be used in connection with the systems and methods described herein. In some examples, AAV production can be induced by infection with a helper virus, such as Ad5, e.g., in the N culture.

Any cell line suitable for use in AAV production can be used as an AAV production host cell with the systems and methods described herein, including suitable mammalian and insect cell lines (including Sf9 cells). For example, a mammalian cell line comprising one or more components needed for AAV production, e.g., a PCL derived from any mammalian cell line, can be used in any of the systems and methods described herein. In some embodiments, the systems and methods described herein comprise culturing mammalian cells, e.g., human cells or non-human mammalian cells, e.g., mammalian PCLs, e.g., human PCL or non-human mammalian PCLs. Exemplary types of cells include but are not limited to: BALB/c mouse myeloma line (NSO/1, ECACC No: 85110503); human retinoblasts (PER.C6 (CruCell, Leiden, The Netherlands)); monkey kidney CVI line transformed by SV40 (COS-7, ATCC CRL 1651); human embryonic kidney line (293 or 293 cells subcloned for growth in suspension culture, Graham et ah, J. Gen Virol., 36:59 (1977)); baby hamster kidney cells (BHK, ATCC CCL 10); Chinese hamster ovary cells+/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CVI ATCC CCL 70); African green monkey kidney cells (VERO-76, ATCC CRL-1 587); human cervical carcinoma cells (HeLa, ATCC CCL 2 or HeLa S3, ECACC Catalog No. 87110901); canine kidney cells (MDCK, ATCC CCL 34); buffalo rat liver cells (BRL 3 A, ATCC CRL 1442); human lung cells (W138, ATCC CCL 75); human liver cells (Hep G2, HB 8065); mouse mammary tumor (MMT 060562, ATCC CCL51); TRI cells (Mather et ah, Annals N.Y. Acad. Sci., 383:44-68 (1982)); FS4 cells: a human hepatoma line (Hep G2); and/or a PCL version of any of the cell types described herein. In some embodiments, a PCL version of a cell type described herein comprises the cell type having been engineered to possess one or more components needed for AAV production.

In some embodiments, the systems and methods described herein comprise culturing Hela cells (e.g., HeLa PCLs), CHO cells (e.g., CHO PCLs), or HEK cells (e.g., HEK PCLs). In some embodiments, the systems and methods described herein comprise culturing HeLa cells, e.g., HeLa PCLs.

As noted above, in some instances the cells will be selected or engineered to include one or more components necessary for AAV production (e.g., engineered into PCLs). Alternatively or additionally, engineered PCLs may comprise genetic modifications to reduce expression and/or activity of one or more genes and/or proteins, which modifications increase AAV titers. Exemplary engineered PCLs useful in the systems and methods of the disclosure include those described in US patent application Publication Number US20200325455A1.

In some embodiments, engineered PCLs may comprise genetic modifications to reduce expression and/or activity of one or more genes and/or proteins, which modifications reduce production or accumulation of waste byproducts in PCLs. As an example of a gene to target for mitigating waste byproduct accumulation, Soo Min Noh et al. showed that shRNA treatment of CHO producer cells to reduce expression of lactate dehydrogenase-A improved production and quality of monoclonal antibodies produced. (See Noh, Soo Min, et al. “Reduction of ammonia and lactate through the coupling of glutamine synthetase selection and downregulation of lactate dehydrogenase-A in CHO cells.” Applied microbiology and biotechnology 101 (3)(2017): 1035-1045, incorporated herein by reference in its entirety.) See also: Shen, Chun Fang, et al. “Reassessing culture media and critical metabolites that affect adenovirus production.” Biotechnology progress 26(1)(2010): 200-207, which describes waste byproduct levels and their effects on Ad5 production: Khajah et al. “Lactate Dehydrogenase A or B Knockdown Reduces Lactate Production and Inhibits Breast Cancer Cell Motility in vitro.” Front Pharmacol. 2021, 12:747001, which shows that knockdown of LDH-A or LDH-B can reduce lactate production; and Prasad et al. “Reduced production and uptake of lactate are essential for the ability of WNT5A signaling to inhibit breast cancer cell migration and invasion.” Oncotarget. 2017 Sep. 22; 8(42): 71471-71488, which shows that PFKP knockdown inhibits lactate production. Each of Shen et al., Khajah et al., and Prasad et al. are incorporated herein by reference in their entireties. Thus, the present disclosure contemplates AAV PCLs engineered to upregulate or downregulate expression of metabolic enzymes that may reduce formation or accumulation of waste byproducts such as lactate or ammonia.

For example, the present disclosure contemplates an engineered AAV PCL expressing glutamine synthetase to reduce lactate and ammonia buildup. Such AAV PCLs may express exogenous glutamine synthetase (GS). Expression of glutamine synthetase may be constitutive or may be inducible. Expression of glutamine synthetase may be driven by a promoter, enhancer, and/or other expression regulatory elements that are optimized for appropriate levels of expression to mitigate waste byproduct buildup during AAV production.

The disclosure also contemplates an engineered AAV PCL having reduced expression of a lactate dehydrogenase to reduce lactate buildup. In some embodiments, the PCLs may have reduced expression of lactate dehydrogenase-A (LDH-A). In some embodiments, the PCLs may have reduced expression of lactate dehydrogenase-B (LDH-B). In some embodiments, the PCLs may have reduced expression of the glycolytic enzyme phosphofructokinase platelet-type (PFKP).

In some embodiments, the PCLs may have increased expression of glutamine synthetase and reduced expression of a lactate dehydrogenase.

Different methodologies for modulating gene expression in a PCL will be recognized by those skilled in the art, and include, for example, approaches set forth in US Patent Application Publication No. US20200325455A1. Examples of methodologies that may be utilized to modulate expression of a gene or gene product in a PCL engineered to reduce production or accumulation of waste byproducts include use of a nuclease, a double stranded RNA (dsRNA), a small interfering RNA (siRNA), a small hairpin RNA (shRNA), a microRNA (miRNA), or an antisense RNA oligonucleotide (ASO). Examples of nucleases include zinc finger nucleases (ZFNs), a meganucleases, transcription activator-like effector nucleases (TALENs), or clustered regularly interspaced short palindromic repeats (CRISPR) associated proteins.

Alternative approaches to reducing lactate and/or ammonia build-up in PCL culture include those described by Freund and Croughan (Int. J. Sci. 2018 February; 19(2): 385). These include alteration of pH, clonal selection of lactate-consumption phenotype, and various media supplementation approaches. It is now further contemplated that media supplementation with essential amino acids (EAAs) may reduce lactate and other waste product build-up and encourage rAAV production by facilitating protein production, based on observations that EAA supplementation in fed-batch culture shifts the metabolic profile of AAV producing cells and in particular reduces lactate build-up (data not shown).

One skilled in the art will appreciate that different cell lines might have different nutrition requirements and/or might require different culture conditions for optimal growth: the person skilled in the art will be able to modify conditions as needed.

### B. Methods for AAV Production

Various aspects of AAV manufacturing are described here, including aspects of the systems and methods described.

i. Batch Mode Process for AAV Production

Generally, in batch mode AAV production, AAV PCLs are cultured in a N−1 culture vessel to achieve a high viable cell density. N−1 culture cells are then used to seed a N culture vessel at a particular seeding density. The seeded cells in the N culture vessel may be cultured under conditions that permit production of AAV.

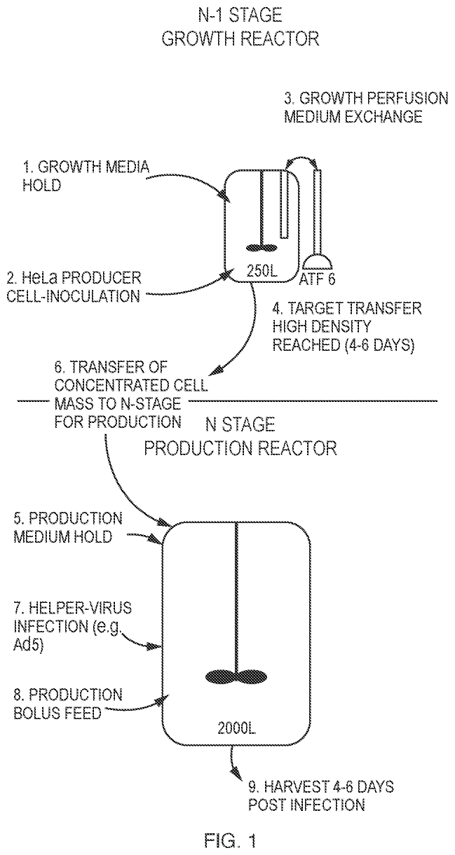

FIG. 1 shows an exemplary process for low density batch (LDB) mode AAV production, i.e., an exemplary batch mode control process. For example, an AAV producer cell line such as a HeLa producer cell line (PCL) is thawed and expanded in shake-flasks, transferred to intermediate scale bioreactors, then transferred to an N−1 perfusion growth vessel at, for example, 250 L scale prior to an production stage production vessel at, for example, 2000 L scale. As shown in FIG. 1, the process begins with growth media being pumped into the 250 L N−1 stage perfusion growth vessel (step 1), followed by seeding the N−1 stage perfusion growth vessel with the preceding N−2 stage producer cell inoculum (step 2). Growth medium is perfusion exchanged (step 3) for, e.g., 4-6 days through use of an Alternating Tangential Flow (ATF) filter system or similar medium exchange system (e.g., Repligen Xcell ATF™ 6 single use alternating tangential flow filtration system at 250 L scale) to achieve a target cell density greater than or equal to 13 E06 vc/mL (viable cells per milliliter)(step 4). For the production stage in batch mode operation, a separate 2000 L vessel is primed with approximately 90% of the end volume of production medium (step 5) and AAV producer cells are transferred from the N−1 to the production stage reactor at a split ratio of approximately 1:10 to enable sufficient exchange of growth medium into production medium (step 6). In FIG. 1, the solid line separating the N−1 stage growth reactor and the N-stage production reactor indicates that these two stages are carried out in separate vessels. In this manner, the 2000 L production vessel is inoculated with the high concentration cell culture (i.e., greater than 13 E06 vc/mL) targeting a final concentration of approximately greater than 1.3 E06 vc/mL. Following inoculation, AAV production is initiated by helper-virus (e.g., adenovirus, Ad5) infection (step 7) and followed with bolus production feed addition to the bioreactor vessel (step 8). After approximately 4-6 days post-infection (“dpi”), AAV product is harvested (step 9) from culture supernatant for subsequent purification and helper virus removal operations. For instance, Ad5 removal processes include those described in, e.g., US patent application Publication Number US20190083554A1.

ii. Modified Batch Systems and Methods Using Perfusion (e.g., Intensified Perfusion) for AAV Production

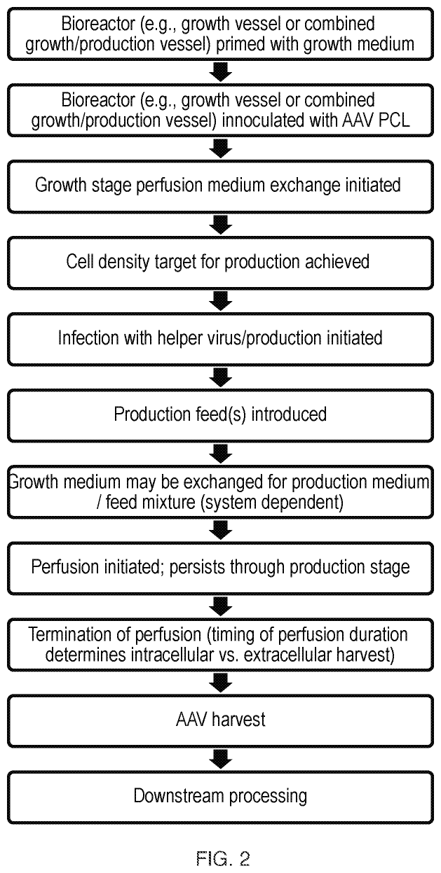

Modified batch processes using perfusion (e.g., intensified perfusion) for AAV production are described herein. An exemplary process is shown in FIG. 2. Generally, modified batch processes involve preparing a vessel with growth medium for AAV producing cell growth stage culture. In some embodiments, the vessel may be a single vessel intended for culturing both growth and production stage cultures, i.e., a “combined growth/production vessel.” Such vessel is subsequently inoculated with AAV producing cells. Perfusion may be initiated in the inoculated growth or combined growth/production vessel to initiate AAV producing cell growth. Once a final cell density target is achieved, production stage culture can be initiated. In some embodiments, production stage initiation involves infecting the growth stage culture with a helper virus to initiate AAV production. AAV production can be initiated by alternative means not dependent on AAV helper virus infection. Such alternative means include, for example, use of triple transfection approaches or use of producer cells with stable integration of components necessary to produce AAV, such as adenovirus helper virus functions, AAV replicase, and capsid genes (e.g., the ELEVECTA® (CEVEC) doxycycline-inducible AAV producing cells, and similar systems). AAV production stage can be maintained, e.g., by providing feed, exchanging medium for production medium, and/or initiating perfusion to sustain production. AAV produced accordingly can be harvested using any suitable separation technology and purified using standard downstream processing steps known to those skilled in the art.

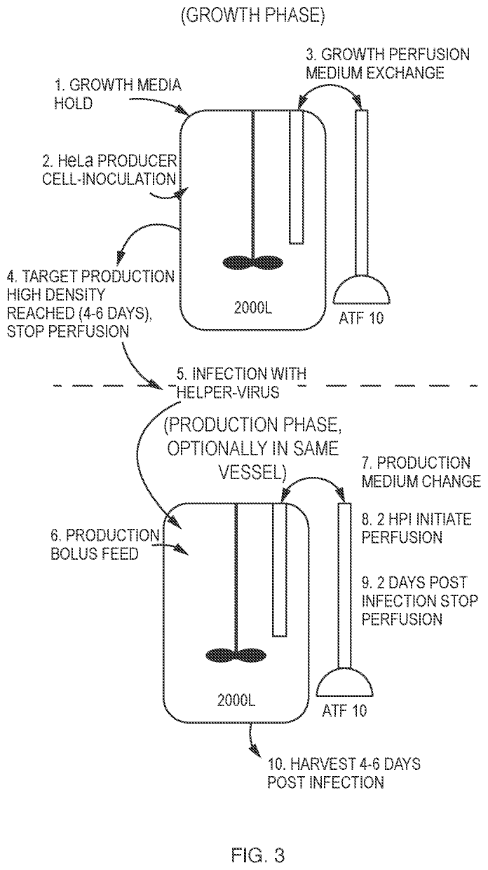

FIG. 3 shows an exemplary process for the AAV production systems and methods described herein. In some embodiments, the preliminary steps for the modified batch (e.g., intensified perfusion) processes may be similar to conventional steps employed in batch mode AAV production processes. For example, the cell seed-train cell mass may be thawed and carried in shake-flasks in the same manner as in batch mode operation.

A bioreactor expansion stage may be utilized to scale up to growth/production culture. Any suitable scale can be employed or adapted to the modified batch systems and methods using perfusion (e.g., intensified perfusion) described herein. In some embodiments, the bioreactor expansion stage uses, e.g., a 250 L scale vessel. In some embodiments, the bioreactor expansion stage uses a sub-liter culture volume. In some embodiments, the bioreactor expansion stage uses any suitable culture volume from sub-liter to 6000 L scale or greater, including up to 12000 L scale. Accordingly, the present systems and methods can utilize a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, or 6000 L vessel in the bioreactor expansion stage culture. In some embodiments, the bioreactor expansion stage uses a vessel with greater than 6000 L capacity. Those skilled in the art will appreciate that larger-scale culture volumes (such as 6000 L or greater) may require specialized media handling systems/apparatuses. Accordingly, the bioreactor expansion stage may utilize an appropriate perfusion apparatus such as an alternating tangential flow (ATF) filtration system. The bioreactor expansion stage culture may involve a perfusion apparatus such as Repligen Xcell ATF™ 6 or other perfusion system suitable to the culture volume selected for the bioreactor expansion stage. At larger-scale culture volumes (such as 6000 L or greater), multiple such apparatuses can be employed, e.g., multiple Repligen Xcell ATF™ 6 or ATF™ 10 systems. In some embodiments, the bioreactor expansion stage does not utilize an ATF filter system.

In some embodiments, the final perfusion production stage is carried out in a production vessel, at, e.g., 2000 L scale. The production vessel may be a vessel suited to any culture volume for AAV production at any scale. For example, the production vessel may be a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, 6000 L, 6500 L, 7000 L, 7500 L, 8000 L, 8500 L, 9000 L, 9500 L, 10000 L, 10500 L, 11000 L, 11500 L, 12000 L vessel, or greater.

In some embodiments, the final perfusion growth and production stages are combined into the same vessel, referred to herein as the combined growth/production vessel, at, e.g., 2000 L scale. The combined growth/production vessel may be a vessel suited to any culture volume for AAV production at any scale. For example, the combined growth/production vessel may be a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, 6000 L, 6500 L, 7000 L, 7500 L, 8000 L, 8500 L, 9000 L, 9500 L, 10000 L, 10500 L, 11000 L, 11500 L, 12000 L vessel, or greater. In some embodiments, the combined growth/production vessel uses a vessel with greater than 6000 L capacity.

Those skilled in the art will appreciate that larger-scale culture volumes (such as 6000 L or greater) may require specialized media handling systems/apparatuses. Accordingly, the growth, production, and/or combined growth/production vessel(s) may utilize an appropriate perfusion apparatus such as an alternating tangential flow (ATF) filtration system. The growth, production, and/or combined growth/production vessel culture(s) may involve a perfusion apparatus such as Repligen Xcell ATF™ 6 or other perfusion system suitable to the culture volume selected for the bioreactor stage. At larger-scale culture volumes (such as 6000 L or greater), multiple such apparatuses can be employed, e.g., multiple Repligen Xcell ATF™ 6 or ATF™ 10 systems or flat-sheet Tangential Flow Filtration systems. For instance, Repligen Xcell ATF™ 6 or comparable a comparable system may be used at, e.g., 50 L-500 L scale, whereas ATF™ 10 or a comparable system may be used at larger scales. In some embodiments, to support increased cell density, multiple ATF™ 10 systems or the like can be implemented. In some embodiments, the growth, production, and/or combined growth/production vessel does not utilize an ATF filter system.

In some embodiments, a perfusion system such as an ATF filter system such as Repligen Xcell ATF™ 10 is implemented in the growth and/or production stages in the combined growth/production vessel. Alternative perfusion technologies suitable for use in the combined growth/production vessel include, but are not limited to, Xcellerex Automated Perfusion System (APS)(Cytiva), KrosFlo® KPS TFF Systems, e.g., KPS 700, or XCell™ ATF 6 (Repligen), GEA Kytero® Single Use Pharma Separator systems (GEA), Prostak™ Microfiltration Modules (Millipore), Alfa Laval CultureOne™ (Alfa Laval), CARR® Centritech Separation Systems such as CARR UniFuge® Pilot or Centritech CELL 8® (Pneumatic Scale Angelus), Ksep® 6000S System (Sartorius), a microfluidic cell retention device (as described in Kwon T. et al. Sci Rep 7, 6703 (2017)), SciLog® SciPure™ systems (Parker), acoustic settler systems (as described in Coronel J. et al. Front. Bioeng. Biotechnol., 2 Jul. 2020)(Sonosep), and/or EDO Electro systems (as described in Wang, Zhaowei, “Two Approaches for Cell Retention in Perfusion Culture Systems” (2009). ETD Archive. 304)(American Piezo). A variety of formats may be available from the vendors for the foregoing perfusion technologies and may be suitable for use in the systems and methods described herein. For example, Repligen ATF systems in 2, 4, 6, or 10 filter unit formats can be implemented, depending on culture scale and cell density desired. Smaller-scale perfusion technologies such as WAVE bioreactors (Cytiva) can also be utilized. Alternative media exchange technologies can be implemented, including tangential flow filtration (TFF) and/or centrifugation-based technology.

In some embodiments, the N-stage growth culture may target a final density of at least 5 E06 vc/mL. The N-stage growth may target any final density suitable for seeding a production stage AAV bioreactor, including, for example, 1 E06, 2 E06, 3 E06, 4 E06, 5 E06, 6 E06, 7 E06, 8 E06, 9 E06, 10 E06, 20 E06, 30 E06, 40 E06, or up to 50 E6 vc/mL, or greater.

In some embodiments, the growth stage culture may target a final density using, e.g., a blend of growth and production medium that is perfused over the course of, e.g., 2-14 days. In some embodiments, only growth medium is used in the growth stage culture. In some embodiments, only production medium is used in the growth stage culture. In some embodiments, a 50/50 blend of growth and production medium is used in the growth stage culture. In other embodiments, a 10/90, 20/80, 30/70, or 40/60 blend of growth and production medium is used in the growth stage culture. In still other embodiments, a 90/10, 80/20, 70/30, or 60/40 blend of growth and production medium is used in the growth stage culture. The growth medium, production medium, or blend of growth and production medium can be perfused over the course of, e.g., 2-14 days. In some embodiments, growth medium alone is used until a final density target is achieved, at which point production medium alone is used throughout the remainder of the production stage.

With reference to the example process depicted in FIG. 3, as a target cell density is achieved in the growth stage(s), growth medium is primed in the combined growth/production vessel (step 1). AAV producer cells such as any suitable PCL, e.g., Hela cells (e.g., HeLa PCL), are inoculated from the preceding bioreactor expansion stage at the target final density for seeding (step 2). The perfusion rate is then increased with cell mass to maintain a constant cell specific perfusion rate (CSPR) for a duration of about 2 days, about 3 days, about 4 days, about 5 days, about 6 days, or greater than 6 days (step 3). In some embodiments, the perfusion rate is increased with cell mass to maintain constant CSPR for a duration of about 2 days to about 6 days, such as about 2 days to about 3 days, about 2 days to about 4 days, about 2 days to about 5 days, about 2 days to about 6 days, about 3 days to about 4 days, about 3 days to about 5 days, about 3 days to about 6 days, about 4 days to about 5 days, about 4 days to about 6 days, or about 5 days to about 6 days. In some embodiments, maintaining a constant CSPR minimizes nutrient depletion and waste accumulation.

In some embodiments, a fixed volumetric perfusion rate is utilized irrespective of cell density.

Once the target cell density is reached for AAV production (e.g., 5 E06 vc/mL)(step 4), the growth perfusion exchange may be halted. AAV production may then be initiated by helper virus (e.g., adenovirus Ad5) infection (step 5). In FIG. 3, the dashed line separating the growth stage and the production stage reactor indicates that both stages can be, but need not be, carried out in the same vessel, e.g., a 2000 L production stage bioreactor. In some embodiments, helper virus infection is followed by bolus feed addition to support production stage activity (step 6). In some embodiments, growth medium is then swapped for production medium/feed mixture (step 7) in, e.g., the ATF-10 set-up. In some embodiments, perfusion is reinitiated approximately 1 hour to approximately 2 hours post infection (hpi)(step 8). Perfusion may be maintained for 1-2 dpi to enrich the medium, reduce accumulation of waste products, and retain AAV product intracellularly. In some embodiments, AAV particles remain cell-associated/intracellular until approximately 48 hpi. As such, to minimize product loss to the waste permeate stream, perfusion may be disabled before this time. After approximately 1-2 dpi, perfusion is disabled to allow retention and accumulation of AAV particles in the culture supernatant (step 9). After about 48 hours, most AAV product is produced but remains intracellular: but from 2-6 dpi, AAV particles are preferentially released into the cell culture supernatant and can be harvested (step 10). In some embodiments, AAV product can be harvested beginning at approximately 2 dpi, approximately 3 dpi, approximately 4 dpi, approximately 5 dpi, approximately 6 dpi, or later than 6 dpi. For example, AAV product may be harvested at about 2 dpi to about 3 dpi, about 2 dpi to about 4 dpi, about 2 dpi to about 5 dpi, about 2 dpi to about 6 dpi, about 3 dpi to about 4 dpi, about 3 dpi to about 5 dpi, about 3 dpi to about 6 dpi, about 4 dpi to about 5 dpi, about 4 dpi to about 6 dpi, or about 5 dpi to about 6 dpi for subsequent downstream purification.

In some embodiments, Ad5 removal processes include those described in, e.g., US patent application Publication Number US20190083554A1.

Those skilled in the art will appreciate that, in both batch mode operation and in modified batch mode operation (e.g., intensified perfusion) as described herein, it will be important to optimize growth and production culture conditions to improve AAV production yield. Each of temperature, pH, osmolality, salt concentration, media and feed composition, oxygen saturation, agitation rate, starting glucose/glutamine concentrations, waste carry over levels, split ratio, culture age, state of cell growth/synchronization, helper virus type or mutation/attenuation, and multiplicity of infection may impact AAV productivity in a particular system or process. Further, waste or metabolite production/consumption rates and media exchange rates may need to be optimized for high-yielding high cell density AAV production processes.

The Examples section of the present application demonstrates exemplary embodiments of the modified batch systems and methods using perfusion (e.g., intensified perfusion) described herein which consistently increase infection density at time of AAV production approximately 4-fold from existing optimal low-density batch (LDB) processes, to achieve a 2.5 fold to 3-fold increase in AAV volumetric productivity of greater than or equal to 3 E11 GC/mL (genome copies per milliliter) or greater than or equal to 3 E14 GC/L.

The modified batch systems and methods using perfusion (e.g., intensified perfusion) described herein have been assessed in the context of two separate HeLa producer cell line systems that utilize different growth media, different genes of interest for therapeutic payloads, and different capsids and have achieved similar fold-improvements in productivity relative to corresponding low-density batch mode operation conditions. These results demonstrate that the modified batch systems and methods using perfusion (e.g., intensified perfusion) described herein can be implemented to achieve AAV yield benefits across multiple AAV producing cell systems, under different media conditions, with differing genes of interest, and differing AAV capsids.

The following sections a.-j. provide examples of various AAV culture parameters that can be utilized in various embodiments of the modified batch systems and methods using perfusion (e.g., intensified perfusion) described herein.

a. Media Exchange, Perfusion

In some embodiments, culturing AAV PCLs in the modified batch systems and methods using perfusion (e.g., intensified perfusion) disclosed herein comprises removing waste from culture media and/or supplementing media with nutrients, e.g., by using perfusion, e.g., alternating tangential flow (ATF) perfusion in the growth stage and/or production stage culture vessel. Perfusion can be utilized to build cell mass, e.g., in a growth stage culture, and/or to maintain cell mass in a production stage culture.

ATF employs a filter system that provides a way to continuously or semi-continuously exchange the cell culture media, e.g., by continuously/semi-continuously supplementing the culture with fresh media while removing waste, e.g., through a multitude of hollow fibers. The ATF flow rate indicates how fast the cell culture circulates through each hollow fiber. In some embodiments, the diameter of each fiber is about 0.5 mm to about 1.5 mm, e.g., about 0.5 mm, about 1 mm, or about 1.5 mm. Without wishing to be bound by theory, it is believed that a higher flow rate tends to increase the amount of shear stress on the cells. And, too low of a flow rate may increase (1) the risk of the cell culture being exposed to low oxygen environments for a prolonged period of time and (2) the possibility of filter fouling due to insufficient back flushing. The methods described herein comprise performing the ATF perfusion at a flow rate that minimizes shear stress on the cells yet provides sufficient oxygen to the cells. In some embodiments, the ATF perfusion is performed at a flow rate of about 3 mL/min/fiber to about 15 mL/min/fiber, e.g., about 5 mL/min/fiber to about 10 mL/min/fiber, about 5 mL/min/fiber to about 8 mL/min/fiber, about 7 mL/min/fiber to about 10 mL/min/fiber, or about 8 mL/min/fiber to about 10 mL/min/fiber. In some embodiments, the ATF perfusion is performed at a flow rate of about 3 mL/min/fiber, about 4 mL/min/fiber, about 5 mL/min/fiber, about 6 mL/min/fiber, about 7 mL/min/fiber, about 8 mL/min/fiber, about 9 mL/min/fiber, about 10 mL/min/fiber, about 11 mL/min/fiber, about 12 mL/min/fiber, about 13 mL/min/fiber, about 14 mL/min/fiber, or about 15 mL/min/fiber. In other embodiments, the ATF perfusion is performed at a flow rate per fiber of about 50 meters/s to about 320 meters/s, e.g., about 50 meters/s to about 250 meters/s, about 50 meters/s to about 200 meters/s, about 100 meters/s to about 320 meters/s, about 100 meters/s to about 250 meters/s, about 200 meters/s to about 320 meters/s, or about 200 meters/s to about 275 meters/s.

In some embodiments, perfusion rate can be optimized to maintain shear rate to increase AAV product release. Shear stress on cells introduced by perfusion can result in rupture of cells and/or release of AAV particles. Accordingly, perfusion rates can be optimized to maintain a shear rate that results in constant release of AAV product in a perfusion culture. AAV released in the culture in this manner can be collected at various timepoints or constantly during production stage, for example from the permeate as free AAV particles pass through the perfusion membrane.

In the first 12 hours, 24 hours, 36 hours, 48 hours, 60 hours, 72 hours, 84 hours, or 96 hours of AAV production, media exchange can be accomplished using any suitable perfusion-based or other media exchange technology, including ATF as described above. Alternative perfusion technologies suitable for use in various embodiments of the systems and methods described herein include Xcellerex Automated Perfusion System (APS)(Cytiva), KrosFlo® KPS TFF Systems, e.g., KPS 700, or XCell™ ATF 6 (Repligen), GEA Kytero® Single Use Pharma Separator systems (GEA), Prostak™ Microfiltration Modules (Millipore), Alfa Laval CultureOne™ (Alfa Laval), CARR® Centritech Separation Systems such as CARR UniFuge® Pilot or Centritech CELL 8® (Pneumatic Scale Angelus), Ksep® 6000S System (Sartorius), a microfluidic cell retention device (as described in Kwon T. et al. Sci Rep 7, 6703 (2017)), SciLog®. SciPure™ systems (Parker), acoustic settler systems (as described in Coronel J. et al. Front. Bioeng. Biotechnol., 2 Jul. 2020)(Sonosep), and/or EDO Electro systems (as described in Wang, Zhaowei, “Two Approaches for Cell Retention in Perfusion Culture Systems” (2009). ETD Archive. 304)(American Piezo). For example, Repligen ATF systems in 2, 4, 6, or 10 filter unit formats can be implemented, depending on scale and cell density. Alternative media exchange technologies can be implemented, including tangential flow filtration (TFF) and/or centrifugation-based technology.

Higher cell densities may require higher perfusion, media exchange, and/or waste removal rates in order to maintain a certain cell growth rate or viability. Cell density can change over time in a cell culture. Thus, a cell specific perfusion rate, a parameter that quantifies the perfusion rate based on the cell density of the culture, may be employed in the systems and methods described herein. In some embodiments, the modified batch systems and methods using perfusion (e.g., intensified perfusion) described herein comprise performing perfusion in the growth stage and/or production stage culture at a cell specific perfusion rate (CSPR) of about 0.02 nL/cell/day to about 0.1 nL/cell/day, e.g., about 0.02 nL/cell/day to about 0.08 nL/cell/day, about 0.02 nL/cell/day to about 0.06 nL/cell/day, about 0.03 nL/cell/day to about 0.08 nL/cell/day, about 0.03 nL/cell/day to about 0.06 nL/cell/day, e.g., about 0.03 nL/cell/day, about 0.04 nL/cell/day, about 0.05 nL/cell/day, or about 0.06 nL/cell/day. In some embodiments, the modified batch systems and methods using perfusion (e.g., intensified perfusion) described herein comprise performing perfusion in the growth stage and/or production stage culture at a CSPR of about 100 pL/cell/day to about 2000 pL/cell/day, said range including all integers in and between 100 pL/cell/day to 2000 pL/cell/day. For instance, the CSPR may be about 100 pL/cell/day to about 1500 pL/cell/day, about 100 pL/cell/day to about 1000 pL/cell/day, about 100 pL/cell/day to about 900 pL/cell/day, about 100 pL/cell/day to about 800 pL/cell/day, about 100 pL/cell/day to about 700 pL/cell/day, about 100 pL/cell/day to about 600 pL/cell/day, about 100 pL/cell/day to about 500 pL/cell/day, about 100 pL/cell/day to about 400 pL/cell/day, about 100 pL/cell/day to about 300 pL/cell/day, about 100 pL/cell/day to about 200 pL/cell/day. In some embodiments, the CSPR may be about 150 pL/cell per day to about 750 pL/cell/day, such as about 150 pL/cell/day, about 200 pL/cell/day, about 250 pL/cell/day, about 300 pL/cell/day, about 350 pL/cell/day, about 400 pL/cell/day, about 450 pL/cell/day, about 500 pL/cell/day, about 550 pL/cell/day, about 600 pL/cell/day, about 650 pL/cell/day, about 700 pL/cell/day, or about 750 pL/cell/day. It will be understood that CSPR in a given system will vary depending on various parameters including, for example, cell density.

In some embodiments, the modified batch systems and methods using perfusion (e.g., intensified perfusion) described herein comprise performing perfusion in the growth stage and/or production stage culture at a constant perfusion rate irrespective of cell density.

b. Cell Culture Media and Supplementation

The cell culture(s) described herein may be prepared in any medium suitable for the particular cell being cultured. In some embodiments, the medium contains, e.g., inorganic salts, carbohydrates (e.g., sugars such as glucose, galactose, maltose or fructose), amino acids, vitamins (e.g., B group vitamins (e.g., B 12), vitamin A, vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum, newborn calf serum and horse serum), trace elements (e.g., zinc, copper, selenium and tricarboxylic acid intermediates), hydrolysates (hydrolyzed proteins derived from plant or animal sources), and combinations thereof. Exemplary suitable cell culture media include minimum essential medium (MEM) such as Eagle's culture medium, Dulbecco's modified Eagle's medium (DMEM), minimum essential medium alpha (MEM-alpha), mesenchymal cell basal medium (MSCBM), Ham's F-12 medium and Ham's F-10 medium, DMEM/F12 medium, William's medium E, RPMI-1640 medium, MCDB medium, medium 199, Fisher's medium, Iscove's modified Dulbecco's medium (IMDM), and McCoy's modified medium. In addition, any of the media described in Ham R., McKechan W., (1979) Meth. Enzymol, 58:44-93; Barnes and Sato, (1980) Anal. Biochem., 102 (2):255-70; U.S. Pat. Nos. 4,657,866, 4,767,704, 4,927,762, 5,122,469 and 4,560,655; or International Publication Nos. WO 90/03430 and WO 87/00195 can be used as culture media in the presently disclosed systems and methods. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range) lipids (such as linoleic or other fatty acids) and their suitable carriers, and glucose or an equivalent energy source. In some embodiments, the nutrient media is serum-free media, a protein-free media, or a chemically defined media. Any other necessary supplements can also be included at appropriate concentrations that would be known to those skilled in the art.

In some embodiments, culturing AAV PCLs in the growth stage and/or production stage culture vessel (or the combined growth/production vessel) comprises supplementing with nutrients, e.g., using a fed batch culture process comprising supplementing the culture periodically with a supplement (e.g., fresh media, amino acids, and/or glucose). In some embodiments, the supplementation occurs at least once every two days, e.g., once a day, twice a day, three times a day, four times a day, or more. In further embodiments, the supplementation occurs about once a day. In some embodiments, the supplement comprises amino acids, e.g., a mixture of one or more amino acids. In some embodiments, the supplement comprises glutamine. In some embodiments, the supplement comprises glucose. In some embodiments, the supplement comprises glucose and glutamine. In some embodiments, the concentration of supplement added each time is determined based on the viable cell density, e.g., viable cell density determined at the time of supplement addition or just prior to supplement addition. In some embodiments, the amount (e.g., volume and/or concentration) of the supplement is determined based on the integrated cell growth (ICG), e.g., in the growth stage and/or production stage culture. ICG can be calculated as described, e.g., in International Patent Application Publication Number WO2020/154607.

In some embodiments, the supplement comprises a total amino acid concentration of about 50 mM to about 2 M. In certain embodiments, the supplement comprises a total amino acid concentration of at least 50 mM. In some embodiments, the supplement comprises a total amino acid concentration of about 75 mM to about 500 mM. In some embodiments, the supplement comprises a total amino acid concentration of about 50 mM to about 1 M, about 50 mM to about 500 mM, about 50 mM to about 100 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 500 mM, about 750 mM, about 800 mM, about 1 M, about 1.5 M, or about 2 M.

In some embodiments, nutrient concentrations are maintained at a desired level and/or waste products are maintained at a low level, e.g., during the growth stage culture, e.g., during any day of the growth stage culture, or during the production stage culture. In some embodiments, the concentration of glucose, glutamine, glutamate, total amino acid, lactate, and/or ammonia in the N−1 culture is maintained at a level within 10-fold (e.g., within 10-fold, 9-fold, 8-fold, 7-fold, 6-fold, 5-fold, 4-fold, 3-fold, 2-fold, or less) of the concentration on day 0 of the growth stage and/or production stage culture. In some embodiments, the concentration of any of these molecules is measured, e.g., on any day of the culture, e.g., using standard methods.

c. Vessel Volumes Per Day

In some embodiments, the modified batch systems and methods using perfusion (e.g., intensified perfusion) described herein comprise culturing the cells during growth stage and/or production stage at a medium exchange rate expressed in terms of vessel volumes of medium per day (VVD). The growth stage culture may be carried out at a medium exchange rate of approximately 1 VVD to about 10 VVD. For example, the growth stage culture may be carried out at a medium exchange rate of 1 VVD, 1.5 VVD, 2 VVD, 2.5 VVD, 3 VVD, 3.5 VVD, 4 VVD, 4.5 VVD, 5 VVD, 5.5 VVD, 6 VVD, 6.5 VVD, 7 VVD, 7.5 VVD, 8 VVD, 8.5 VVD, 9 VVD, 9.5 VVD, or 10 VVD. Likewise, the production stage culture may be carried out at a medium exchange rate of approximately 1 VVD to about 10 VVD. For example, the production stage culture may be carried out at a medium exchange rate of 1 VVD, 1.5 VVD, 2 VVD, 2.5 VVD, 3 VVD, 3.5 VVD, 4 VVD, 4.5 VVD, 5 VVD, 5.5 VVD, 6 VVD, 6.5 VVD, 7 VVD, 7.5 VVD, 8 VVD, 8.5 VVD, 9 VVD, 9.5 VVD, or 10 VVD. In some embodiments, the growth and production stage culture(s) are carried out at a medium exchange rate of between 1 VVD, 1.5 VVD, 2 VVD, 2.5 VVD, 3 VVD, 3.5 VVD, 4 VVD, 4.5 VVD, 5 VVD, 5.5 VVD, 6 VVD, 6.5 VVD, 7 VVD, 7.5 VVD, 8 VVD, 8.5 VVD, 9 VVD, 9.5 VVD, or 10 VVD.

d. Cell Density and Viability

In some embodiments, the modified batch systems and methods using perfusion (e.g., intensified perfusion) described herein comprise culturing the cells to a target cell density (e.g., expressed in terms of viable cell density) of at least 1 E06 viable cells/mL (vc/mL), at least 1 E07 vc/mL, at least about 1 E08 vc/mL, or at least about 1 E09 vc/mL. In some embodiments, the methods described herein comprise culturing the cells in the growth stage culture to a target cell density of about 1 E06 vc/mL to about 50 E06 vc/mL, e.g., about 1 E06 vc/mL, about 2 E06 vc/mL, about 3 E06 vc/mL, about 4 E06 vc/mL, about 5 E06 vc/mL, 6 E06 vc/mL, 7 E06 vc/mL, 8 E06 vc/mL, 9 E06 vc/mL, 10 E06 vc/mL, 20 E06 vc/mL, 30 E06 vc/mL, 40 E06 vc/mL, or 50 E06 vc/mL. In some embodiments, the methods described herein comprise culturing the cells in the growth stage culture to a target cell density of about 1 E06 vc/mL, about 1.1 E06 vc/mL, about 1.2 E06 vc/mL, about 1.3 E06 vc/mL, about 1.4 E06 vc/mL, about 1.5 E06 vc/mL, about 1.6 E06 vc/mL, about 1.7 E06 vc/mL, about 1.8 E06 vc/mL, about 1.9 E06 vc/mL, about 2 E06 vc/mL, about 2.1 E06 vc/mL, about 2.2 E06 vc/mL, about 2.3 E06 vc/mL, about 2.4 E06 vc/mL, about 2.5 E06 vc/mL, about 2.6 E06 vc/mL, about 2.7 E06 vc/mL, about 2.8 E06 vc/mL, about 2.9 E06 vc/mL, about 3 E06 vc/mL, about 3.1 E06 vc/mL, about 3.2 E06 vc/mL, about 3.3 E06 vc/mL, about 3.4 E06 vc/mL, about 3.5 E06 vc/mL, about 3.6 E06 vc/mL, about 3.7 E06 vc/mL, about 3.8 E06 vc/mL, about 3.9 E06 vc/mL, about 4 E06 vc/mL, about 4.1 E06 vc/mL, about 4.2 E06 vc/mL, about 4.3 E06 vc/mL, about 4.4 E06 vc/mL, about 4.5 E06 vc/mL, about 4.6 E06 vc/mL, about 4.7 E06 vc/mL, about 4.8 E06 vc/mL, about 4.9 E06 vc/mL, about 5 E06 vc/mL, or greater than 5 E06 vc/mL. In some embodiments, the methods described herein comprise culturing the cells in the growth stage culture to a target cell density of about 1 E06 vc/mL to about 1 E07 vc/mL, about 1 E07 vc/mL to about 1 E08 vc/mL, or about 1 E08 vc/mL to about 1 E09 vc/mL. In some embodiments, the methods described herein comprise culturing the cells in the growth stage culture to a target cell density of about 1 E06 vc/mL to about 1.5 E06 vc/mL, about 1.5 E06 vc/mL to about 2 E06 vc/mL, about 2 E06 vc/mL to about 2.5 E06 vc/mL, about 2.5 E06 vc/mL to about 3 E06 vc/mL, about 3 E06 vc/mL to about 3.5 E06 vc/mL, about 3.5 E06 vc/mL to about 4 E06 vc/mL, about 4 E06 vc/mL to about 4.5 E06 vc/mL, or about 4.5 E06 vc/mL to about 5 E06 vc/mL.

In some embodiments, the methods described herein comprise determining the viable cell density periodically, e.g., at least once every 3 days, once every 2 days, once a day, twice a day, or more frequently, e.g., once per minute, two times per minute, three times per minute, four to ten times per minute, once per hour, twice per hour, three times per hour, four to ten times per hour, once per second, two times per second, three times per second, four times per second, five times per second, six times per second, seven times per second, eight times per second, nine times per second, or ten times per second.

e. Cell Growth Rate

In some embodiments, the cell growth rate is maintained at a growth rate that is close to the maximum growth rate for the cell line/clone being cultured. The systems and methods described herein may achieve a growth rate within 15% (e.g., within 15%, 12.5%, 10%, 7.5%, 5%, 2.5%, 1%, or less) of the maximum growth rate of the cell line/clone being cultured.

In some embodiments, the maximum growth rate is the growth rate of the particular cell line/clone measured while in fresh culture medium and during its exponential growth phase (e.g., measured at a point in time when nutrients are sufficient and no components in the culture are causing significant growth inhibition). In some embodiments, the overall growth rate depends on the particular cell type/clone being cultured. In some embodiments, the overall growth rate of the cells is about 0.2/day to about 1/day, e.g., about 0.3/day to about 0.8/day, about 0.4/day to about 0.7/day, about 0.5/day to about 0.7/day, about 0.4/day to about 0.6/day, about 0.2/day to about 0.8/day, or about 0.5/day to about 1/day. In some embodiments, the overall growth rate is determined based on the cell density at day zero of the culture.

f. pH

In some embodiments of the modified batch systems and methods using perfusion (e.g., intensified perfusion) described herein, the pH of the growth stage and/or production stage culture is maintained, e.g., between 5 and about 9, e.g., about 6.4 to about 8.2. For example, in some embodiments, the pH of the growth stage culture is maintained at about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, or about 8.2. In some embodiments, the pH of the production stage culture is maintained at about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, or about 8.2.

g. Dissolved Oxygen

In some embodiments of the modified batch systems and methods using perfusion (e.g., intensified perfusion) described herein, the dissolved oxygen (DO) levels are maintained for optimal growth and or AAV production in the growth stage and/or production stage culture. For example, the DO levels may be maintained at 10% to 100%, for instance at maintained 50% set-point.

h. Culture Duration

In some embodiments, the AAV producing cells are cultured in the growth stage and/or production stage culture for 4 days to 12 days, e.g., 4 days, 5 days, 6 days, 7 days, 8 days, 9 days, 10 days, 11 days, 12 days, or 13 days. In some embodiments, the production stage culture is maintained for a duration of 2 or more days, e.g., 2 days, 3 days, 4 days, 5 days, 6 days, 7 days, 8 days, 9 days, 10 days, 11 days, 12 days, 13 days, or 14 days.

In some embodiments, the production stage culture is maintained under conditions that permit the production of AAV. In some embodiments, one or more components of AAV are added in the production stage culture such that the cells in the production stage culture are replication-competent and can produce AAV. In some embodiments, a helper virus, e.g., an adenovirus (e.g., an Ad5 helper virus) or a herpes virus, is added in the production stage culture.

In certain embodiments, the methods comprise culturing the cells in the production stage culture for less than a week, e.g., less than 7 days, e.g., less than 6 days, e.g., 5 days, 4 days, 3 days, 2 days, 1 day or less, e.g., 2-5 days, e.g., 2, 3, 4, or 5 days.

i. Culture Volume

In some embodiments, the growth stage culture is carried out any appropriate scale for AAV producing cell growth phase. For example, the growth stage culture may be carried out at sub-liter to 6000 L scale. Accordingly, the present systems and methods can utilize a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, or 6000 L vessel in the bioreactor for growth stage culture.

In some embodiments, any pre-production stage culture is carried out any appropriate scale for AAV producing cell growth phase. For example, any pre-production stage culture may be carried out at sub-liter to 6000 L scale. Accordingly, the present systems and methods can utilize a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, or 6000 L vessel in the bioreactor for pre-production or growth stage culture.

In some embodiments, the production stage culture is carried out any appropriate scale for AAV production. For example, the production stage culture may be carried out at sub-liter to 6000 L scale. Accordingly, the present systems and methods can utilize a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, or 6000 L vessel in the bioreactor for production stage culture.

In some embodiments, the growth and production stage culture(s) is/are carried out in a combined growth/production vessel at any appropriate scale for AAV growth and production. For example, the growth/production stage culture(s) may be carried out in the same combined growth/production vessel at sub-liter to 6000 L scale. Accordingly, the present systems and methods can utilize a sub-liter, 1 L, 2 L, 5 L, 10 L, 20 L, 50 L, 100 L, 150 L, 200 L, 250 L, 500 L, 1000 L, 1500 L, 2000 L, 2500 L, 3000 L, 3500 L, 4000 L, 4500 L, 5000 L, 5500 L, or 6000 L vessel in the bioreactor for production stage culture.

j. Downstream Processes

In some embodiments, the methods described herein further comprise collecting or harvesting AAV product from the production stage culture. Standard methods of collecting or separating viral particles can be used, e.g., including but not limited to filtration or centrifugation. Further downstream purification processes can be utilized to purify collected AAV product.

In accordance with the methods described herein, the AAV titer, e.g., in the N culture, can be determined, e.g., on any one or more days of the N culture. An exemplary AAV titer measurement is vector genome copies per cell (GC/cell). GC/cell can be determined by standard methods in the art, e.g., including but not limited to dot blot, quantitative PCR or ddPCR, spectroscopy, or fluorimetry. See, e.g., Dorange et al. Cell Gene Therapy Insights 4.2 (2018): 119-129.

In some embodiments, the methods described herein are capable of producing an AAV titer, e.g., in the production stage culture, of at least about 1 E09 vg/mL, at least about 2 E09 vg/mL, at least about 3 E09 vg/mL, at least about 4 E09 vg/mL, at least about 5 E09 vg/mL, at least about 6 E09 vg/mL, at least about 7 E09 vg/mL, at least about 8 E09 vg/mL, at least about 9 E09 vg/mL, at least about 1 E10 vg/mL, at least about 2 E10 vg/mL, at least about 3 E10 vg/mL, at least about 4 E10 vg/mL, at least about 5 E10 vg/mL, at least about 6 E10 vg/mL, at least about 7 E10 vg/mL, at least about 8 E10 vg/mL, at least about 9 E10 vg/mL, at least about 1 E1l vg/mL, at least about 2 E11 vg/mL, at least about 3 E11 vg/mL, at least about 4 E11 vg/mL, at least about 5 E11 vg/mL, at least about 6 E11 vg/mL, at least about 7 E11 vg/mL, at least about 8 E11 vg/mL, at least about 9 E11 vg/mL, at least about 1 E12 vg/mL, at least about 2 E12 vg/mL, at least about 3 E12 vg/mL, at least about 4 E12 vg/mL, at least about 5 E12 vg/mL, or greater.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition, system, or method described herein can be combined in a variety of ways without departing from the spirit and scope of the present disclosure, whether explicit or implicit herein. For example, where reference is made to a particular compound or system component, that compound or system component can be used in various embodiments of compositions/systems of the present disclosure and/or in methods of the present disclosure, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings. For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with two or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions in a system or process is immaterial so long as the object of the present disclosure remains operable. Moreover, two or more steps or actions may be conducted simultaneously in many embodiments of the systems and methods described herein.

The use of any and all examples, or exemplary language herein, for example, “such as” or “including” is intended merely to illustrate better the presently disclosed inventions and does not pose a limitation on the scope of the inventions unless claimed. No language in the specification should be construed as indicating any non-claimed element as essential to the practice of the presently disclosed systems and methods for AAV production.

### EXAMPLES

The inventive systems and methods now being generally described, will be more readily understood by reference to the following examples, which are included merely for purposes of illustration of certain aspects and embodiments of the present disclosure, and are not intended to limit the disclosure.

### Materials and Methods

Metabolites (glucose, L-glutamine, ammonia, and lactate) were evaluated in AAV producer cell culture by Nova Bio-profile Flex2 (Nova Biomedical). Cell density was evaluated in AAV producer cell culture by Vi-Cell XR (Beckman-Coulter). AAV titer was quantified by DNase Resistant Particle (DRP) analysis by quantitative DNA polymerase chain reaction (qPCR) for specific AAV gene therapy product(s). Adenovirus byproduct was also evaluated by DRP-qPCR for specific adenoviral gene(s), e.g., E2A.

### Example 1: Increasing Cell Density in Batch Mode Alone is not Sufficient to Increase AAV Volumetric Yield

High cell density production was tested in batch AAV production mode to determine if increasing cell density could improve AAV volumetric yield. It was determined that increasing cell density in batch mode operation negatively impacts AAV productivity.

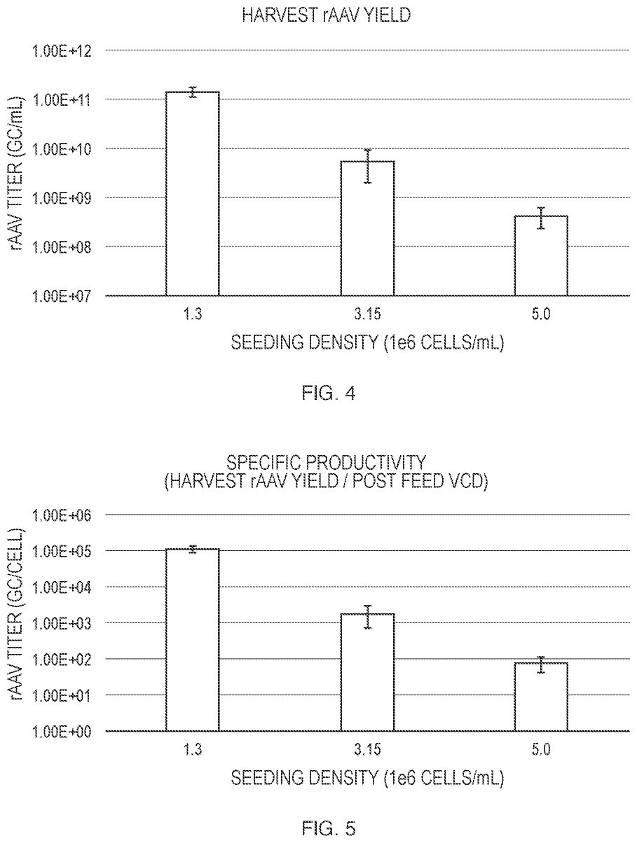

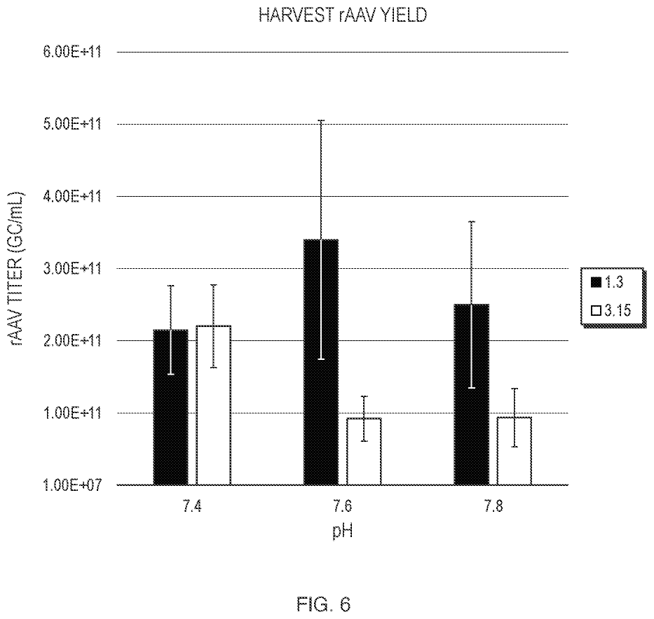

HeLa producer cells were inoculated in bioreactors (2 L w.v.) at 1.3 E06 viable cells per milliliter (vc/mL), 3.15 E06 vc/mL, or 5.0 E06 vc/mL and infected with adenovirus type 5 (Ad5) to initiate AAV production for a gene therapy product in batch mode operation. The AAV capsid was a clade E capsid, AAV hu37. Batch mode conditions in this process utilized a 1.3 E06 vc/mL target seeding density. Increasing the cell density 2 fold and 4 fold (to 3.15 E06 and 5 E06 vc/mL) led to approximately 10- and 100-fold reduction in volumetric rAAV yield (FIG. 4) and cell specific rAAV productivity (FIG. 5), respectively.

Rapid depletion of nutrients such as glucose and L-glutamine was observed with significant increase in waste byproduct (such as lactate and ammonia). It was thus hypothesized that nutrient depletion and/or waste build-up led to reduced AAV productivity (data not shown).

This example demonstrates that batch AAV production methods cannot support AAV production at higher cell densities. Indeed, increasing cell density in batch mode resulted in over 100-fold decrease in AAV yield with a 4-fold increase in seeding density for AAV production.

### Example 2: Reducing pH at Higher Cell Density in Batch Mode Increases AAV Productivity

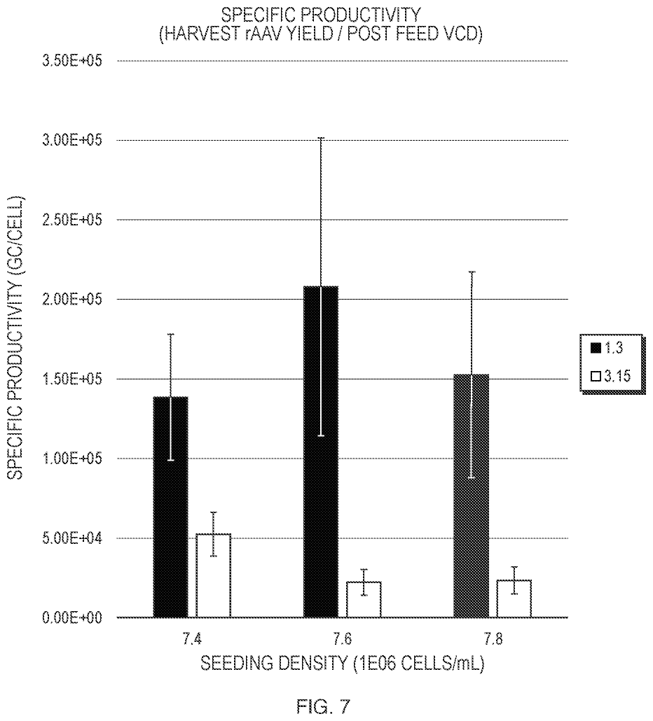

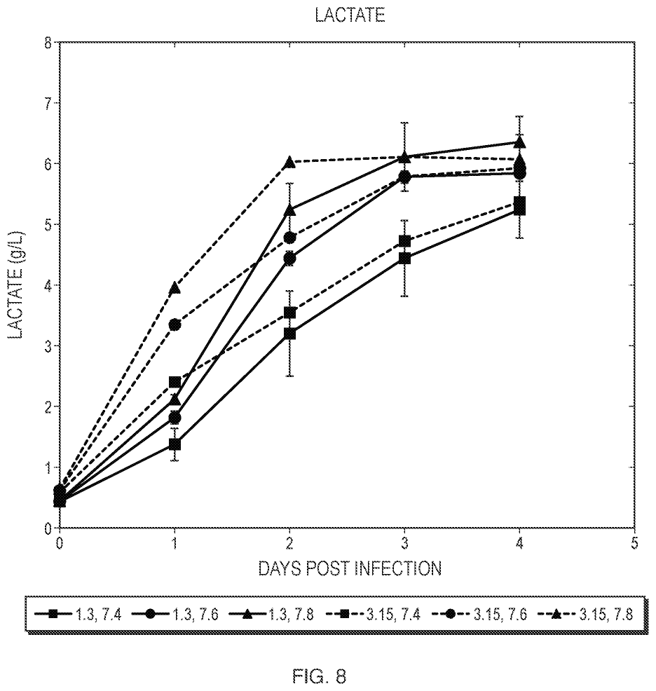

Attempts were made to improve AAV productivity at higher density in batch mode (e.g., higher than about 1 E06 vc/mL). It was determined that reducing pH at higher cell density increases AAV productivity and reduces waste build up in batch mode. HeLa producer cells were inoculated in bioreactors (2 L w.v.) at 1.3 E06 vc/mL or 3.15 E06 vc/mL and infected with Ad5 to initiate AAV production in batch mode operation with a pH set-point of pH 7.4, 7.6, or 7.8. In batch mode at 1.3 E06 vc/mL, a pH of 7.6 to 7.8 showed highest rAAV yield (though not significantly different from yield seen at pH 7.4)(FIG. 6 and FIG. 7, solid bars); however, at a cell density of 3.15 E06 vc/mL, a pH of 7.4 showed higher AAV volumetric (FIG. 6, empty bars) and specific productivity (FIG. 7, empty bars) yield. At higher cell density (3.15 E06 vc/mL) and low pH (7.4), AAV productivity was increased, which coincided with reduced waste byproduct accumulation (lactate) (FIG. 8). This suggests that AAV productivity may be inhibited by waste byproduct accumulation. Process and medium/feed modifications to reduce accumulation of waste byproducts (e.g., lower pH to lower lactate levels) and/or increased nutrient utilization may lead to improved AAV production capability in higher cell density culture.

### Example 3: AAV and Adenovirus Cellular Release Dynamics Enable Perfusion Capability

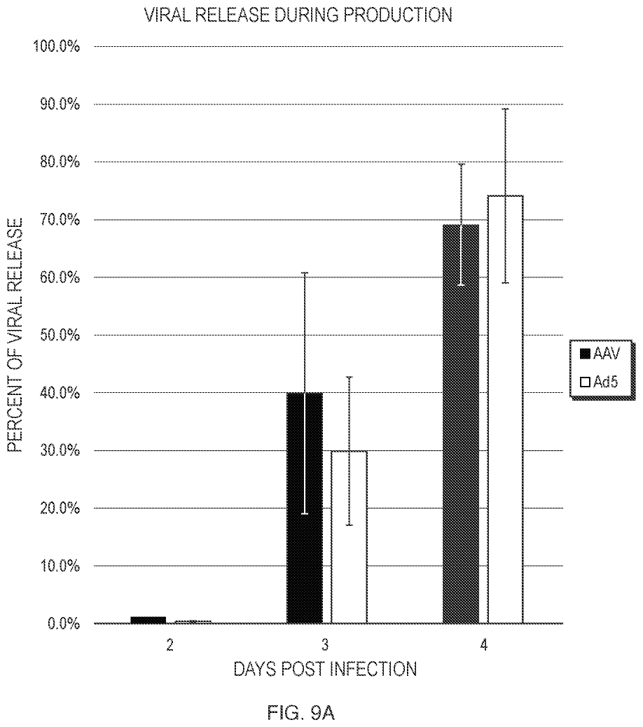

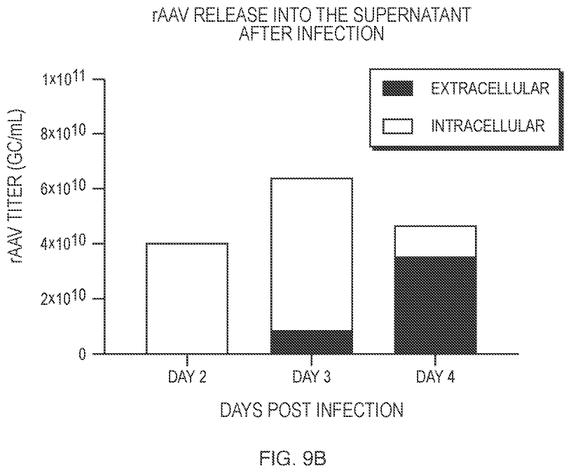

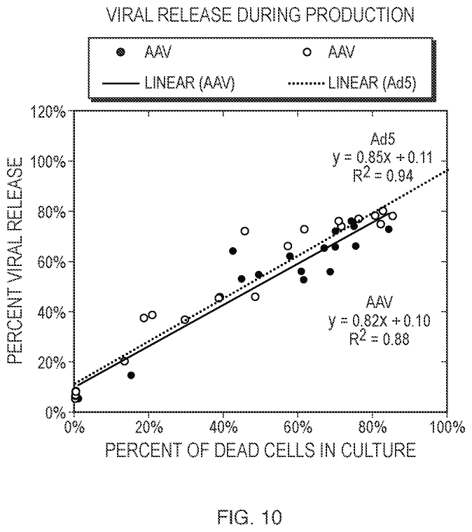

AAV and Ad5 titer and cell viability were monitored over time in several 2 L bioreactor (AAV, n=17 and Ad5, n=25) batch mode runs post infection with adenovirus. It is known that cellular infection with adenovirus induces apoptosis through E1A, E3, and E4 genes, resulting in cell lysis (see, e.g., Jiang, Hong, et al. J. Virology 85.10 (2011): 4720-4729 and Chinnadurai, G. Seminars in VIROLOGY. Vol. 8. No. 5. Academic Press, 1998). Plotting % viral release vs. days post infection, it was observed that for the first 2 days post-infection, the majority of AAV and adenovirus remain associated with the intracellular fraction (FIG. 9A). From day 2 to day 3 to day 4, approximately 30% increase in non-cellular associated (i.e., free) AAV and adenovirus particles per day was observed. Plotting rAAV titer vs. days post infection (FIG. 9B) illustrated the start of release of rAAV into the supernatant after infection. Additionally, a strong correlation of AAV and Ad5 release with cell death during production was observed (FIG. 10), suggesting that viral release results from cell lysis. The AAV and adenovirus release dynamics are important to understand when to initiate and how long to maintain perfusion to maximize product yield and recovery. Based on the above findings from HeLa producer cells (Examples 1 and 2), the optimal perfusion start timing based on adenovirus cellular uptake at different densities was identified to be approximately between 1 to 2 hpi (data not shown). In addition, perfusion was maintained for up to 48 hpi to avoid significant product losses due to perfusion media exchange, as the majority of AAV remains intracellular for the first 2 days post infection with helper virus.

These results also demonstrate that the optimal harvest can extend beyond 4 days to support maximum product recovery from culture supernatant, as only 70%-80% of AAV is released after 4 days post-infection and it may be important to extend the harvest to 5 days, 6 days, 7 days, etc. or longer, depending on the specific optimized culture AAV dynamics.

### Example 4: Perfusion Process Optimization of Media Exchange Rate

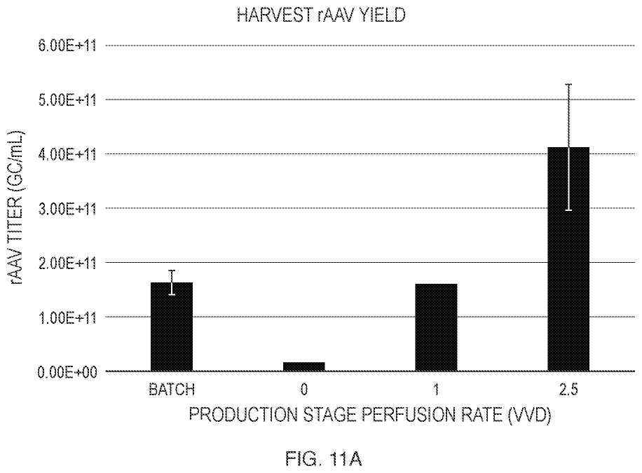

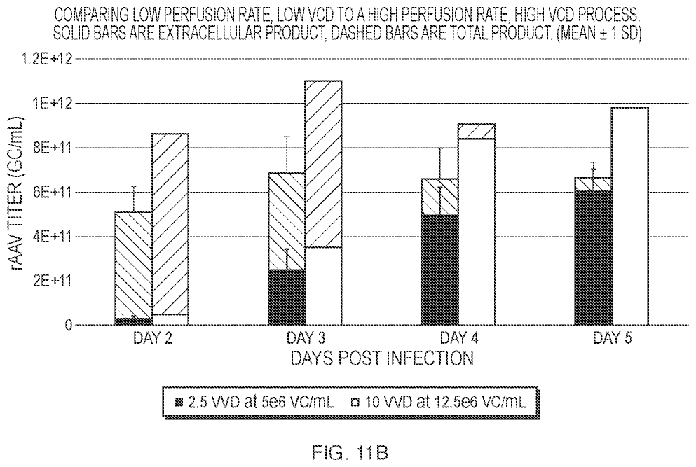

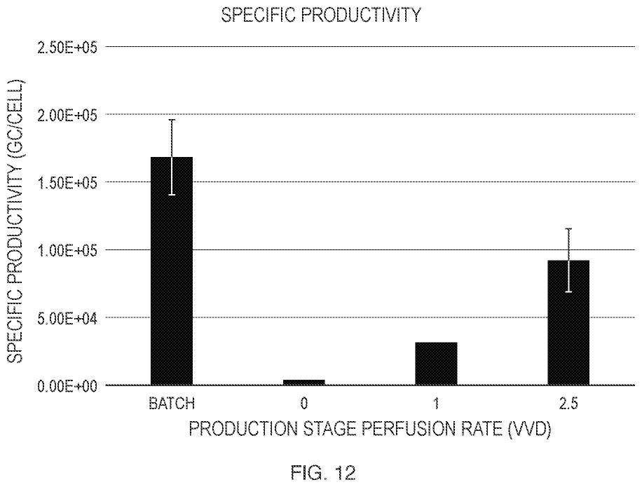

In batch mode operation, the cell specific perfusion rate (CSPR) at 3 E06 vc/mL and 5 E06 vc/mL was found to be between 0.5 nL/cell/day to 0.6 nL/cell/day, based on nutrient consumption and waste accumulation rates (data not shown). To evaluate perfusion mode operation for modified batch AAV production at increased cell density, HeLa producer cells were inoculated at 5 E06 vc/mL in 2 L bioreactors with XCell alternating tangential flow 2 (Repligen, ATF™ 2) systems and infected with adenovirus helper virus to initiate AAV production. Two hours post infection, perfusion was initiated with production medium/feed mixture perfusion media exchange rates at either 0, 1 vessel volume per day, or 2.5 vessel volumes per day (VVD) for 48 hours post infection and compared to a batch mode control process. An increase in rAAV volumetric yield (FIG. 11A) and specific productivity (FIG. 12) with increasing VVD was observed. The highest levels were observed at 2.5 VVD with approximately 3-fold increase in volumetric yield and approximately 60% cell specific productivity as compared to the batch mode control process. Therefore, higher VVD conditions were tested up to 10 VVD. Specifically, volumetric productivity was evaluated at 10 VVD with a cell density of 12 E06 cells/mL to 13 E06 cells/mL. Compared to 2.5 VVD conditions, volumetric productivity increased under the increased perfusion rate and cell density conditions (FIG. 11B), with increases in extracellular rAAV product observed compared to the 2.5 VVD conditions at each of Day 2 through Day 5.

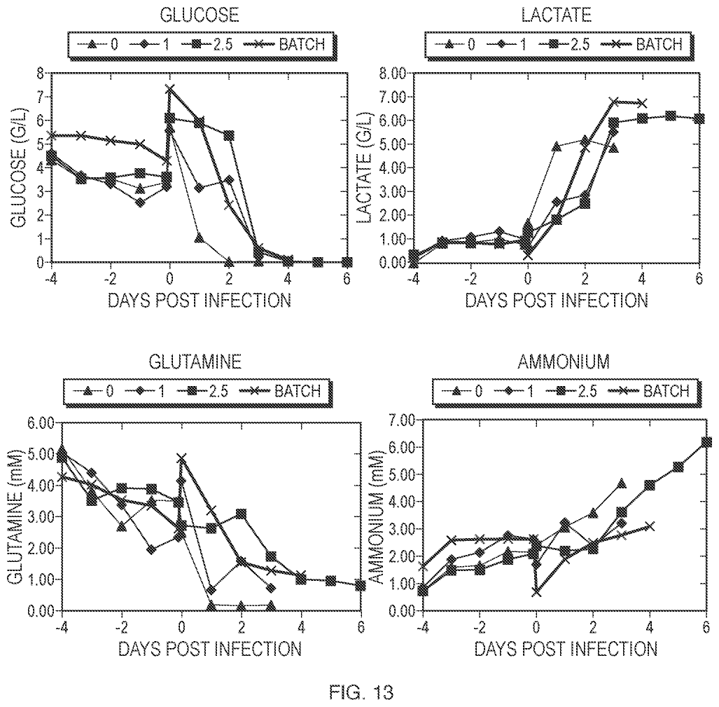

Glucose and glutamine levels rapidly depleted in the high cell density culture with either 0 or 1VVD but were maintained at 2.5 VVD for the 48 hours during perfusion exchange, similar to the batch mode control process (FIG. 13, left two graph panels). Lactate levels increased significantly in the 0 VVD condition, but 1 and 2.5 VVD conditions matched batch mode control (FIG. 13, top right graph panel). All ammonium concentrations were higher in the high density perfusion conditions but remained lower in the 2.5 VVD condition as compared to the 0 and 1 VVD conditions (FIG. 13, bottom right graph panel), indicating that by reducing waste accumulation and nutrient depletion through perfusion medium exchange, between 2-48 hpi, the modified batch process can enable high cell density production conditions in an AAV production system using HeLa producer cells and helper virus.

These data show that increasing perfusion media exchange rate improved cell specific AAV productivity at high density and increased rAAV volumetric yields.

### Example 5: Scale-Up of Modified Batch Process to 50 L

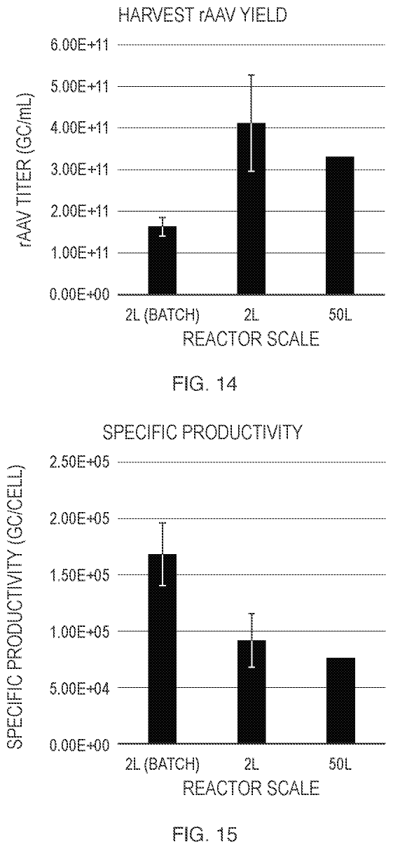

AAV production was performed using modified batch process at 2 L scale using ATF2 and 50 L scale using ATF6 at scaled operating parameters and productivity was compared to the batch mode control process at 2 L scale. AAV volumetric productivity was maintained at 2 L and 50 L scale using the modified batch process and achieved approximately 2.3-fold improvement in volumetric yield over the batch mode control (FIG. 14)(3.3 E11 GC/mL with modified batch process vs. 1.4 E11 GC/mL with batch mode process), with cell specific productivity of approximately 60% (FIG. 15) compared to the batch mode process.

These data demonstrate that the modified batch process for AAV production is scalable and consistently yields fold-improvement in volumetric yield despite modest reduction in specific productivity.

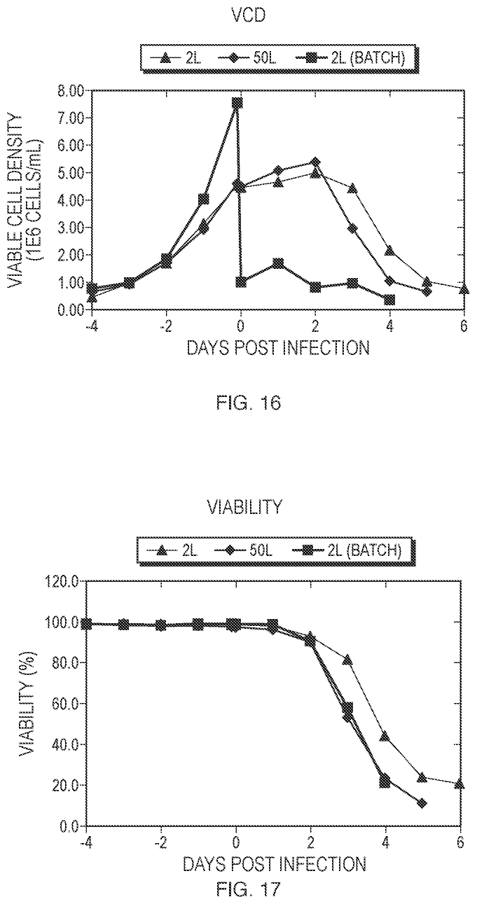

As shown in FIG. 16, viable cell density (VCD) is increased in modified batch processes during production phase as compared to batch mode control process at 1, 2, 3, and 4 dpi. Cell viability profiles are similar in the 2 L and 50 L modified batch process compared to the batch mode control process (FIG. 17).

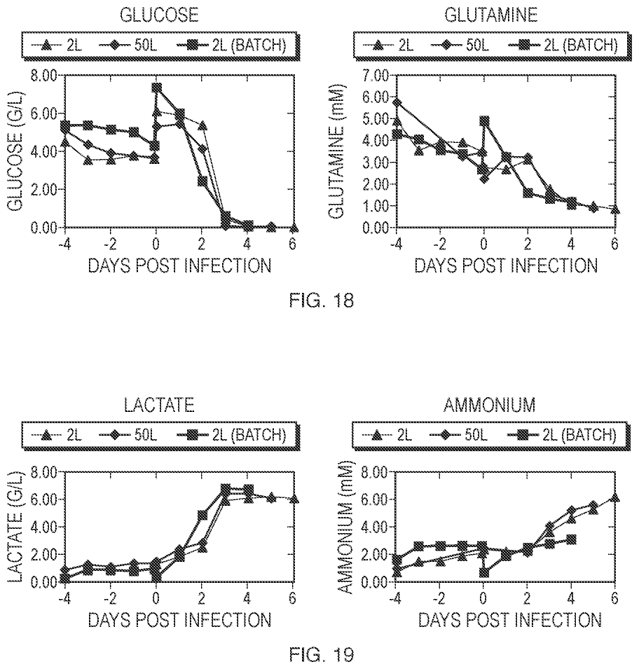

Metabolite analysis demonstrates that the modified batch process maintains similar nutrient and waste levels as compared to the batch mode control process. For instance, glucose and L-glutamine levels are sustained at similar levels through 2 dpi compared to the batch process (FIG. 18), and buildup of lactate and ammonium are comparable to the batch process (FIG. 19). These data demonstrate that the modified batch process enables increased viable cell density in a scalable manner compared to batch mode control processes through maintaining key nutrient consumption and waste accumulation rates, resulting in higher AAV productivity per volume.

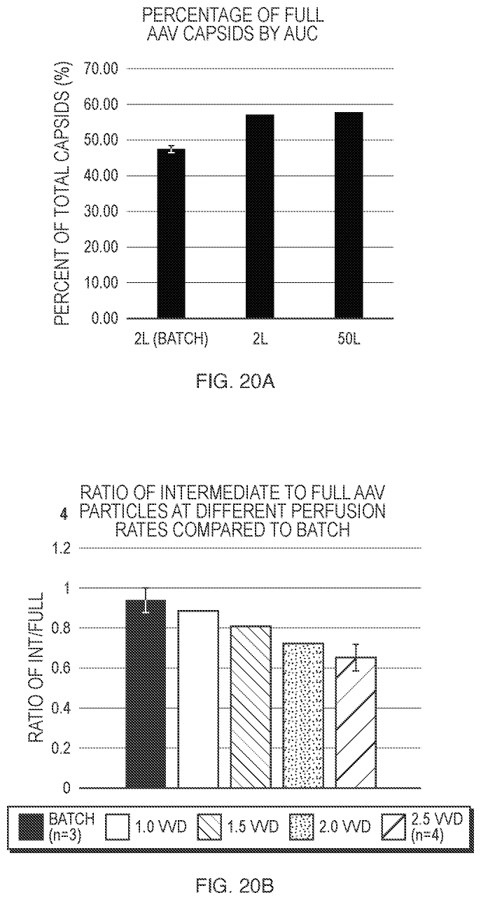

Further, as shown in FIG. 20A, the percent of total capsids determined to contain a full vector genome as assessed by AUC analysis was found to be consistent in 2 L and scaled modified batch processes, showing that the modified batch process does not compromise AAV packaging efficiency. In fact, further analysis demonstrated packaging efficiency utilizing the modified batch process described herein improved with increased VVD. As shown in FIG. 20B, the ratio of intermediate-to-full AAV particles reduced with increasing VVD using the modified batch process as compared to a control batch production process. The observed enrichment in full species and decrease in intermediate species as VVD was increased suggesting additional waste removal/nutrient enrichment is beneficial for AAV product quality.

### Example 6: Modified Batch AAV Production Process is Universal and can be Implemented with Different Media, Vector Genomes/Genes of Interest, and AAV Capsid Serotypes

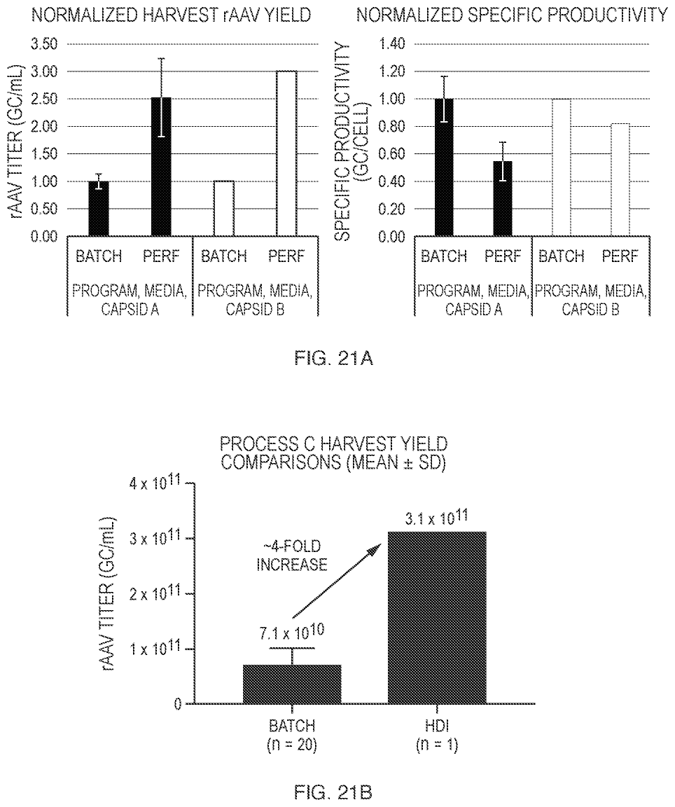

The modified batch process was developed and optimized using program A, media composition A, and capsid A (a clade E capsid, AAV hu37) and compared to a different gene of interest (program B) with different capsid serotype B (clade E, AAV8), and different media composition B. In both processes, A and B, 2.3 fold-3-fold boost in AAV volumetric productivity was observed (FIG. 21A, left graph panel), indicating that our technology could be applied universally to other AAV processes, media and culture conditions and other capsid serotypes. With process A, an approximately 60% cell specific productivity as compared to the batch mode control was observed and with process B an approximately 80% cell specific productivity was observed (FIG. 21A, right graph panel), indicating that optimization in process parameters and media composition may lead to higher yielding production conditions that more closely match the batch mode AAV cell specific production rates.

The modified batch process was further tested using a different program (program C) having a different gene of interest from programs A and B, a different media composition from programs A and B, and a different capsid (a clade F capsid, AAV9) from programs A and B; results were compared to the batch mode process. With the modified batch process, a starting cell density of infection was increased from 1.17 E6 cells/mL to 4.5 E6 cells/mL. At harvest, yield was increased from 7.1 E10 GC/mL to 3.1 E11 GC/mL, an approximate 4-fold increase using the modified batch process described herein (FIG. 21B). Cell health and metabolite profiles (data not shown) were comparable to those seen in other runs using the modified batch process.

These data show that the modified batch methodology (e.g., intensified perfusion) is applicable to a broad range of products, processes and production conditions with varying media conditions, vector genomes/genes of interest, and AAV capsid serotypes.

### Example 7: Lactate and Ammonia Waste Byproducts Inhibit AAV Production

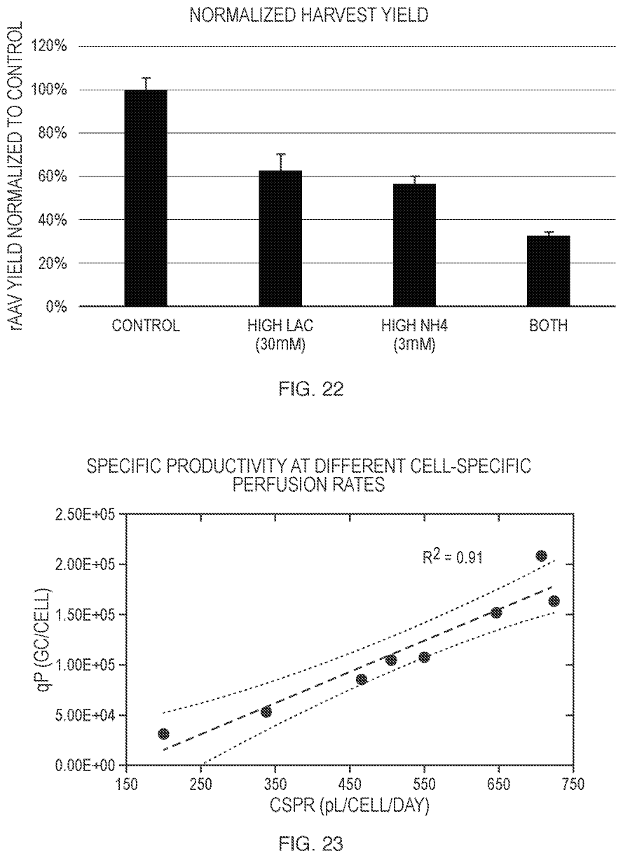

As described in Example 1, high cell density in batch mode AAV production led to significant increase in waste byproduct formation including lactate and ammonia/ammonium. To determine the effects of these waste byproducts on AAV production, production was tested in batch AAV production mode with varying levels of ammonia, lactate, or both. FIG. 22 shows that increased lactate (30 mM)(indicated as “High Lac” in FIG. 22) or increased ammonia (3 mM) (indicated as “High NH4” in FIG. 22) reduced rAAV yield to approximately 60% of control rAAV harvest yield conditions, whereas the addition of both increased lactate and ammonia (indicated as “Both” in FIG. 22) reduced rAAV yield to approximately 35% of control rAAV harvest yield conditions. This example confirms that high cell density AAV production methods require effective waste byproduct removal or mitigation to achieve optimal yield.

### Example 8: Specific Productivity Increases with Increasing CSPR

As shown in FIG. 23, increasing cell-specific perfusion rates from 150 pL/cell/day to 750 pL/cell/day resulted in increased specific productivity (measured in genome copies (GC) per cell). A strong linear correlation of AAV cell specific yield (GC/cell) was observed with CSPR (pL/cell/day) up to approximately 750 pL/Cell/day, R²=0.91. No apparent limit (or plateau) was observed in cell specific productivity with increasing perfusion rate, suggesting that additional increase in CSPR rate greater than 750 pL/Cell/day would continue to increase AAV productivity within our system. These data demonstrate that conditions tested to date have not revealed a plateau in cell specific productivity, illustrating that the modified batch process described herein may be able to achieve further increases in cell specific productivity, perhaps surpassing that seen using batch mode AAV production processes.

### Example 9: Modified Batch AAV Production Process May Significantly Reduce Cost of Goods

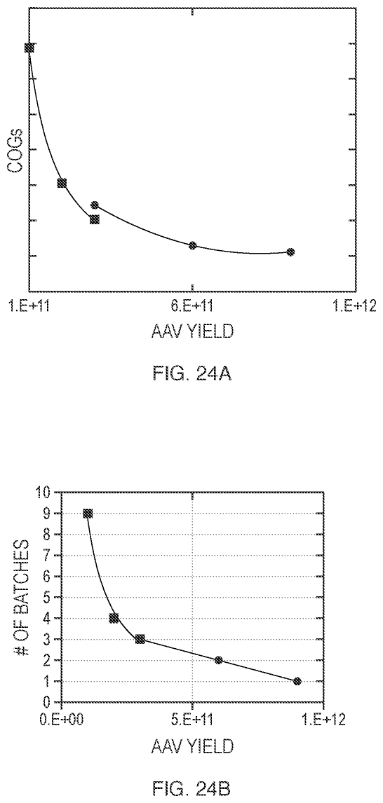

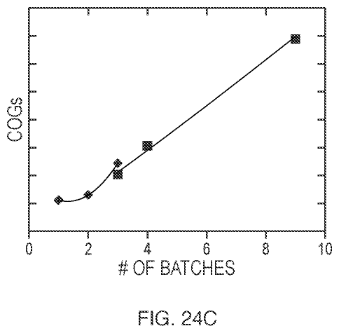

Cost of goods (COGs) projections demonstrate potential to decrease costs associated with AAV production by using the modified batch (e.g., intensified perfusion) production processes described herein. As shown in FIG. 24A-FIG. 24C, even accounting for higher up-front costs per AAV production batch, gains in productivity per volume are projected to be greater than the increased material and operating costs required for operating the modified batch process. As a result, fewer production batches, shorter time frame to generate material, and lower overall COGs can be achieved using the modified batch processes described herein. FIG. 24A shows that approximate projected COGs required per AAV yield are reduced in the modified batch process compared to batch production. FIG. 24B shows that approximate number of production batches required per AAV yield are reduced in the modified batch process compared to batch production. FIG. 24C shows that approximate projected COGs required per number of production batches are reduced in the modified batch process compared to batch production. In FIG. 24A through FIG. 24C, diamonds or circles represents modified batch systems described herein, while squares represent batch operating conditions.

### Example 10: Transition Perfusion Schedule to Replace Blended Growth Media

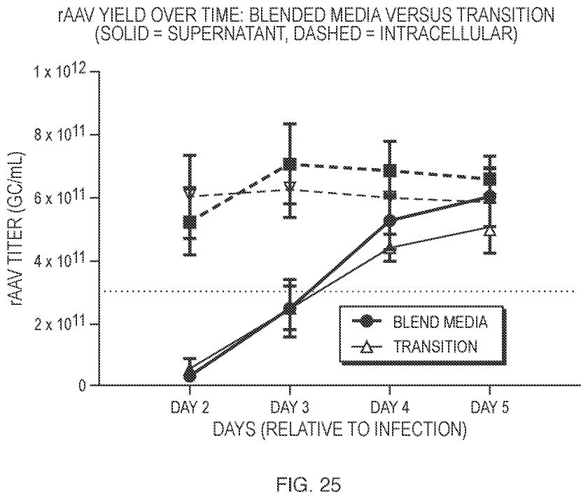

A blended growth and production media was used in the N-growth stage to expand cell density and balance growth and production needs for the cells. However, use of blended media has draw backs, including lack of stability data of the blended media, time required for preparation and general availability of blended media, and higher media consumption during the growth stage. To determine whether a timed transition from growth to production media could avoid the need for a blended media, production media was perfused to the reactor 24 hr prior to infection at 2.5 VVD. No significant difference in rAAV titer was observed between transitioning to production media versus utilization of blended media, either in terms of released or intracellular rAAV (FIG. 25), showing that a transition to perfusion media prior to infection can replace the need for blended media.

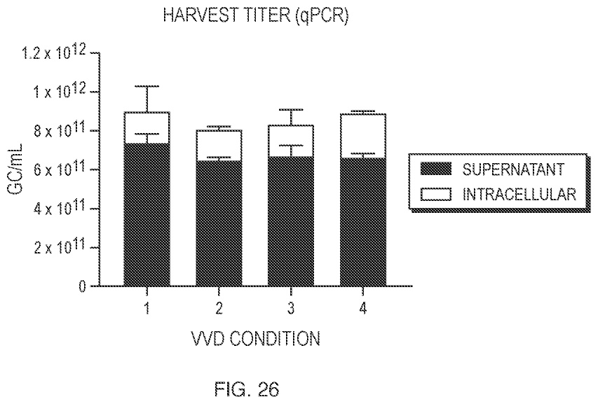

The large volumes of media required for perfusion-based production processes remains a major limitation, despite gains in rAAV productivity. For example, perfusion based production may require 8.5 or more vessel volumes of perfusion media per run, or 17,000 L of media at the 2,000 L production scale. Therefore, to investigate approaches to limit media consumption, reducing the rate of perfusion in the growth stage was tested using the transition perfusion schedule. FIG. 26 shows that the perfusion rate during the growth stage can be reduced to about 0.5 VVD (from about 1 VVD) and the perfusion rate during the transition stage can be reduced to about 1.5 VVD (from about 2.5 VVD), representing a saving of about 3,000 L of media per batch, without losing titer. In FIG. 26, VVD Condition 1 is 1 VVD in the growth stage followed by 2.5 VVD in the transition stage: Condition 2 is 1 VVD in the growth stage and 1.5 VVD in the transition stage; Condition 3 is 0.5 VVD in the growth stage and 2.5 VVD in the transition stage; and Condition 4 is 0.5 VVD in the growth stage and 1.5 VVD in the transition stage.

### Example 11: Scale-Up of Modified Batch Process to 250 L Scale

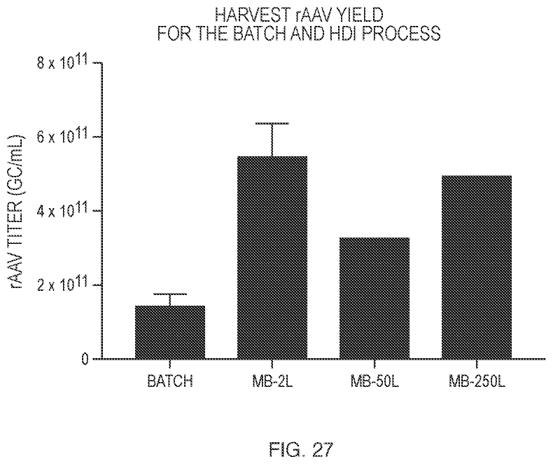

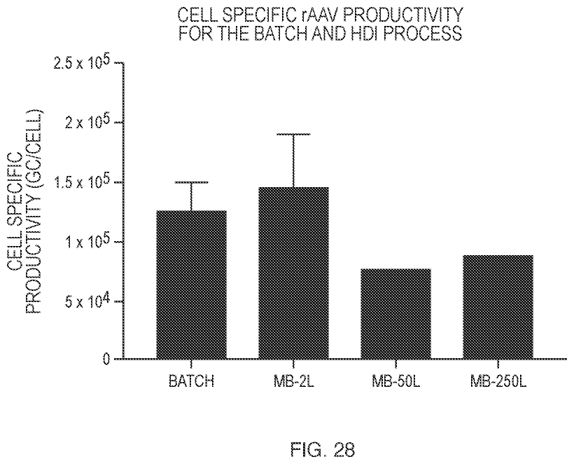

Example 5 shows successful scale-up of the modified batch process to 50 L scale. To further evaluate the scalability of the modified batch rAAV production process, test pilot scale runs were performed at 250 L using ATF6 at scaled operating parameters and productivity was compared to the batch mode control process at 2 L scale (indicated as “Batch” in FIG. 27), the modified batch process at 2 L (indicated as “MB-2 L” in FIG. 27), the modified batch process at 50 L (indicated as “MB-50 L” in FIG. 27), and the modified batch process at 250 L (indicated as “MB-250 L” in FIG. 27). In the 50 L run, blended growth and production media was used in the growth stage. In the 250 L run, 1 VVD perfusion rate was used for growth stage and 2.5 VVD for transition stage. rAAV titer at harvest showed an approximate 3- to 4-fold increase in volumetric yield was achieved using an approximate 3- to 4-fold increase in viable cell density (VCD). As shown in FIG. 27, the modified batch process (abbreviated “MB” in the FIG. 27 and FIG. 28) maintained yield gains at 250 L scale (approximately 5 E11 GC/mL) comparable to the 2 L scale, versus the batch process (<2 E11 GC/mL). As shown in FIG. 28, cell specific productivity was maintained at 250 L scale comparable to that seen at 50 L and 2 L scales.

These data further confirm that the modified batch process for AAV production is scalable and consistently yields fold-improvement in volumetric yield despite modest reduction in specific productivity.

What is claimed is:

1. A method of producing a recombinant adeno-associated virus (rAAV), comprising:
(a) culturing AAV production host cells to a target cell density in a growth vessel using perfusion to supplement the culture with fresh nutrients and/or to remove waste products, wherein the AAV production host cells comprise genetic material encoding one or more AAV components;
(b) initiating expression in the AAV production host cells of step (a) of one or more helper virus functions to initiate rAAV production; and
(c) culturing the AAV production host cells of step (b) in a production vessel using perfusion to supplement the culture with fresh nutrients and/or to remove waste products,
thereby producing the rAAV.

2. The method of claim 1, wherein the AAV production host cells are insect cells or mammalian cells.

3. The method of claim 2, wherein the AAV production host cells are mammalian cells.

4. The method of claim 3, wherein the mammalian cells are Hela cells, Cos-7 cells, HEK293 cells, A549 cells, BHK cells, Vero cells, RD cells, or ARPE-19 cells.

5. The method of any one of claims 1-4, wherein the AAV production host cells comprise an AAV producer cell line (PCL) comprising genetic material encoding one or more AAV components stably integrated into the AAV production host cell genome.

6. The method of any one of claims 1-5, wherein the AAV PCL comprises one or more genetic modifications to reduce expression and/or activity of one or more genes and/or proteins to reduce production or accumulation of lactate and/or ammonia.

7. The method of any one of claims 1-6, wherein the target cell density in step (a) is at least about 1 E06 viable cells/mL (vc/mL).

8. The method of any one of claims 1-7, wherein the target cell density in step (a) is between 1 E06 vc/mL to 5 E07 vc/mL.

9. The method of any one of claims 1-7, wherein the target cell density in step (a) is about 1 E06 vc/mL, about 2 E06 vc/mL, about 3 E06 vc/mL, about 4 E06 vc/mL, about 5 E06 vc/mL, about 6 E06 vc/mL, about 7 E06 vc/mL, about 8 E06 vc/mL, about 9 E06 vc/mL, about 1 E07 vc/mL, about 1.1 E07 vc/mL, about 1.2 E07 vc/mL, about 1.3 E07 vc/mL, about 1.4 E07 vc/mL, about 1.5 E07 vc/mL, about 1.6 E07 vc/mL, about 1.7 E07 vc/mL, about 1.8 E07 vc/mL, about 1.9 E07 vc/mL, or about 2 E07 vc/mL.

10. The method of any one of claims 1-9, wherein the target cell density in step (a) is about 3.15 E06 vc/mL or about 5.0 E06 vc/mL.

11. The method of any one of claims 1-10, wherein the production vessel has a volume between IL to 12000 L.

12. The method of any one of claims 1-11, wherein the production vessel has a IL volume, a 2 L volume, a 5 L volume, a 10 L volume, a 20 L volume, a 50 L volume, a 100 L volume, a 150 L volume, a 200 L volume, a 250 L volume, a 500 L volume, a 1000 L volume, a 1500 L volume, a 2000 L volume, a 2500 L volume, a 3000 L volume, a 3500 L volume, a 4000 L volume, a 4500 L volume, a 5000 L volume, a 5500 L volume, a 6000 L, or a 12000 L volume.

13. The method of any one of claims 1-12, wherein the growth vessel and the production vessel are a combined growth/production vessel.

14. The method of any one of claims 1-13, wherein the one or more AAV components comprise a therapeutic payload or transgene, an inverted terminal repeat (ITR) or two ITRs, one or more AAV replication and/or packaging proteins encoded by the AAV rep gene, and/or one or more AAV structural capsid proteins encoded by the AAV cap gene.

15. The method of any one of claims 1-14, wherein step (b) comprises infecting the cells of step (a) with a helper virus.

16. The method of any one of claims 1-14, wherein step (b) comprises inducing expression of one or more helper virus functions encoded by genetic material in the AAV production host cells.

17. The method of claim 15, wherein the helper virus is an adenovirus.

18. The method of claim 17, wherein the helper virus is Ad5.

19. The method of any one of claims 1-18, wherein the perfusion is performed at a flow rate of about 3 mL/min/fiber to about 15 mL/min/fiber.

20. The method of any one of claims 1-19, wherein the perfusion is performed at a flow rate of about 3 mL/min/fiber, about 4 mL/min/fiber, about 5 mL/min/fiber, about 6 mL/min/fiber, about 7 mL/min/fiber, about 8 mL/min/fiber, about 9 mL/min/fiber, about 10 mL/min/fiber, about 11 mL/min/fiber, about 12 mL/min/fiber, about 13 mL/min/fiber, about 14 mL/min/fiber, or about 15 mL/min/fiber.

21. The method of any one of claims 1-20, wherein the perfusion is performed with a medium exchange rate of about 0.5 vessel volume per day (VVD) to about 10 VVD.

22. The method of any one of claims 1-21, wherein the perfusion is performed with a medium exchange rate of about 0.5 VVD, about 1 VVD, about 1.5 VVD, about 2 VVD, about 2.5 VVD, about 3 VVD, about 3.5 VVD, about 4 VVD, about 4.5 VVD, about 5 VVD, about 5.5 VVD, about 6 VVD, about 6.5 VVD, about 7 VVD, about 7.5 VVD, about 8 VVD, about 8.5 VVD, about 9 VVD, about 9.5 VVD, or about 10 VVD.

23. The method of any one of claims 1-22, wherein the perfusion is performed at a cell-specific perfusion rate (CSPR) of about 150 pL/cell/day to about 2000 pL/cell/day.

24. The method of any one of claims 1-22, wherein the perfusion is performed at a CSPR of about 150 pL/cell/day to about 750 pL/cell/day.

25. The method of any one of claims 1-22, wherein the perfusion is performed at a CSPR of greater than 750 pL/cell/day.

26. The method of any one of claims 1-25, wherein the perfusion is performed using a perfusion system selected from: Xcellerex Automated Perfusion System (APS)(Cytiva); KrosFlo® KPS TFF systems (Repligen); XCell™ ATF systems in 2, 4, 6, 8, or 10 ATF unit formats (Repligen); GEA Kytero® Single Use Pharma Separator systems (GEA); Prostak™ Microfiltration Modules systems (Millipore); Alfa Laval CultureOne™ systems (Alfa Laval); CARR® Centritech Separation Systems CARR UniFuge® Pilot or Centritech CELL 8® systems (Pneumatic Scale Angelus); Ksep® 6000S System (Sartorius); a microfluidic cell retention device; SciLog® SciPure™ systems (Parker); acoustic settler systems; and EDO Electro systems (American Piezo).

27. The method of any one of claims 1-26, wherein step (a) comprises culturing the AAV production host cells in a growth medium.

28. The method of any one of claims 1-27, wherein step (a) and step (c) comprise culturing the AAV production host cells in a growth medium.

29. The method of any one of claims 1-26, wherein step (a) comprises culturing the AAV production host cells in a production medium.

30. The method of claim 29, wherein step (a) and (c) comprise culturing the AAV production host cells in a production medium.

31. The method of any one of claims 15-26, wherein step (a) comprises culturing the AAV production host cells in a growth medium, and step (c) comprises culturing the AAV production host cells in a production medium.

32. The method of claim 31, wherein the perfusion of the production medium is initiated about 1 hour-6 hours after infection of the cells with the helper virus.

33. The method of any one of claims 1-26, wherein step (a) and step (c) comprise culturing the AAV production host cells in a blend of growth and production media.

34. The method of claim 33, wherein the blend of growth and production media is a 10/90, 20/80, 30/70, 40/60, 50/50, 60/40, 70/30, 80/20, or a 90/10 blend of growth and production media.

35. The method of any one of claims 1-26, wherein step (a) comprises culturing the AAV production host cells in a growth medium until about 24 hours prior to step (b), then transitioning to a production medium.

36. The method of claim 35, wherein the growth medium is perfused at a rate of about 0.5 VVD to about 1 VVD until within about 24 hours prior to step (b).

37. The method of claim 35 or 36, wherein the production medium is perfused at a rate of about 1.5 VVD to about 2.5 VVD starting within about 24 hours prior to step (b).

38. The method of any one of claims 1-37, comprising culturing the AAV production host cells in the growth stage and/or production stage culture for about 48 hours, and/or about 2 days to about 14 days.

39. The method of claim 38, comprising culturing the AAV production host cells in the production stage for a duration of about 48 hours, about 2 days, about 3 days, about 4 days, about 5 days, about 6 days, about 7 days, about 8 days, about 9 days, about 10 days, about 11 days, about 12 days, about 13 days, or about 14 days.

40. The method of claim 38 or 39, further comprising halting perfusion after the production stage duration of about 48 hours, about 2 days, about 3 days, about 4 days, about 5 days, about 6 days, about 7 days, about 8 days, about 9 days, about 10 days, about 11 days, about 12 days, about 13 days, or about 14 days.

41. The method of any one of claims 38-40, wherein the rAAV is released from the AAV production host cells into the supernatant of the production medium.

42. The method of claim 41, wherein the rAAV is predominantly released beginning after about 48 hours of infection of the cells with the helper virus.

43. The method of claim 42, wherein the perfusion is stopped at about 48 hours after infection.

44. The method of claim 43, further comprising harvesting the rAAV.

45. The method of claim 44, wherein harvesting the rAAV is performed under batch mode conditions.

46. The method of any one of claims 1-45, further comprising downstream processing of the rAAV.

47. The method of claim 46, wherein the downstream processing comprises purifying, clarifying, and/or concentrating the rAAV.

48. The method of claim 47, wherein the downstream processing comprises filtering cellular debris, colloids, or aggregates.

49. The method of claim 48, wherein the filtering removes particles of cellular debris, colloids, or aggregates more than 1 μm in size.

50. The method of any one of claims 46-49, comprising purifying the rAAV using anion exchange (AEX) chromatography.

51. A method of producing a recombinant adeno-associated virus (rAAV), comprising culturing AAV production host cells using a perfusion system, wherein the AAV production host cell culture has a target cell density between 1 E06 vc/mL to 5 E07 vc/mL.

52. The method of claim 51, wherein the AAV production host cells are insect cells or mammalian cells.

53. The method of claim 52, wherein the AAV production host cells are mammalian cells.

54. The method of claim 53, wherein the mammalian cells are HeLa cells, Cos-7 cells, HEK293 cells, A549 cells, BHK cells, Vero cells, RD cells, or ARPE-19 cells.

55. The method of any one of claims 51-54, wherein the AAV production host cells comprise an AAV producer cell line (PCL) comprising genetic material encoding one or more AAV components stably integrated into the AAV production host cell genome.

56. The method of any one of claims 51-55, wherein the AAV PCL comprises one or more genetic modifications to reduce expression and/or activity of one or more genes and/or proteins to reduce production or accumulation of lactate and/or ammonia.

57. The method of any one of claims 51-56, wherein the target cell density is about 5 E06 vc/mL to about 5 E07 vc/mL.

58. The method of any one of claims 51-57, wherein the target cell density is about 1 E06, about 2 E06, about 3 E06, about 4 E06, about 5 E06, about 6 E06, about 7 E06, about 8 E06, about 9 E06, about 1 E07, about 1.1 E07, about 1.2 E07, about 1.3 E07, about 1.4 E07, about 1.5 E07, about 1.6 E07, about 1.7 E07, about 1.8 E07, about 1.9 E07, or about 2 E07 vc/mL.

59. The method of any one of claims 51-58, wherein the target cell density is about 3.15 E06 vc/mL or about 5.0 E06 vc/mL.

60. The method of any one of claims 51-59, wherein the method is carried out in a production vessel having a volume between 1 L to 12000 L.

61. The method of any one of claims 51-60, wherein the method is carried out in a production vessel having a IL volume, a 2 L volume, a 5 L volume, a 10 L volume, a 20 L volume, a 50 L volume, a 100 L volume, a 150 L volume, a 200 L volume, a 250 L volume, a 500 L volume, a 1000 L volume, a 1500 L volume, a 2000 L volume, a 2500 L volume, a 3000 L volume, a 3500 L volume, a 4000 L volume, a 4500 L volume, a 5000 L volume, a 5500 L volume, a 6000 L, or a 12000 L volume.

62. The method of any one of claims 51-61, further comprising growing the AAV production host cells using the perfusion system to the target cell density in a combined growth/production vessel.

63. The method of any one of claims 51-62, wherein the one or more AAV components comprise a therapeutic payload or transgene, an inverted terminal repeat (ITR) or two ITRs, one or more AAV replication and/or packaging proteins encoded by the AAV rep gene, and/or one or more AAV structural capsid proteins encoded by the AAV cap gene.

64. The method of any one of claims 51-63, further comprising initiating expression of one or more helper virus functions in the AAV production host cells thereby initiating rAAV production.

65. The method of claim 64, wherein initiating expression of one or more helper virus functions comprises infecting the AAV production host cells with a helper virus.

66. The method of claim 64, wherein initiating expression of one or more helper virus functions comprises inducing expression of one or more helper virus functions encoded by genetic material in the AAV production host cells.

67. The method of claim 65, wherein the helper virus is an adenovirus.

68. The method of claim 67, wherein the helper virus is Ad5.

69. The method of any one of claims 51-68, wherein the AAV production host cells are cultured at the target cell density under conditions suitable for the production of rAAV.

70. The method of any one of claims 51-68, wherein the perfusion system operates at a flow rate of about 3 mL/min/fiber to about 15 mL/min/fiber.

71. The method of any one of claims 51-70, wherein the perfusion system operates at a flow rate of about 3 mL/min/fiber, about 4 mL/min/fiber, about 5 mL/min/fiber, about 6 mL/min/fiber, about 7 mL/min/fiber, about 8 mL/min/fiber, about 9 mL/min/fiber, about 10 mL/min/fiber, about 11 mL/min/fiber, about 12 mL/min/fiber, about 13 mL/min/fiber, about 14 mL/min/fiber, or about 15 mL/min/fiber.

72. The method of any one of claims 51-71, wherein the perfusion system operates with a medium exchange rate of about 0.5 vessel volume per day (VVD) to about 10 VVD.

73. The method of any one of claims 51-72, wherein the perfusion system operates with a medium exchange rate of about 0.5 VVD, about 1 VVD, about 1.5 VVD, about 2 VVD, about 2.5 VVD, about 3 VVD, about 3.5 VVD, about 4 VVD, about 4.5 VVD, about 5 VVD, about 5.5 VVD, about 6 VVD, about 6.5 VVD, about 7 VVD, about 7.5 VVD, about 8 VVD, about 8.5 VVD, about 9 VVD, about 9.5 VVD, or about 10 VVD.

74. The method of any one of claims 51-73, wherein the perfusion is performed at a cell-specific perfusion rate (CSPR) of about 150 pL/cell/day to about 2000 pL/cell/day.

75. The method of any one of claims 51-73, wherein the perfusion is performed at a CSPR of about 150 pL/cell/day to about 750 pL/cell/day.

76. The method of any one of claims 51-73, wherein the perfusion is performed at a CSPR of greater than 750 pL/cell/day.

77. The method of any one of claims 51-76, wherein the perfusion system is selected from: Xcellerex Automated Perfusion System (APS)(Cytiva); KrosFlo® KPS TFF systems (Repligen); XCell™ ATF systems in 2, 4, 6, 8, or 10 ATF unit formats (Repligen); GEA Kytero® Single Use Pharma Separator systems (GEA); Prostak™ Microfiltration Modules systems (Millipore); Alfa Laval CultureOne™ systems (Alfa Laval); CARR® Centritech Separation Systems CARR UniFuge® Pilot or Centritech CELL 8® systems (Pneumatic Scale Angelus); Ksep® 6000S System (Sartorius); a microfluidic cell retention device; SciLog® SciPure™ systems (Parker); acoustic settler systems; and EDO Electro systems (American Piezo).

78. The method of any one of claims 51-77, wherein the AAV production host cells are cultured in a growth medium followed by in a production medium.

79. The method of claim 78, wherein the transition to the production medium occurs at about 24 hours prior to infecting the AAV production host cells with a helper virus.

80. The method of claim 78 or 79, wherein the growth medium is perfused at a rate of about 0.5 VVD to about 1 VVD until within about 24 hours prior to infecting the AAV production host cells with the helper virus.

81. The method of any one of claims 78-80, wherein the production medium is perfused at a rate of about 1.5 VVD to about 2.5 VVD starting within about 24 hours prior infecting the AAV production host cells with helper virus.

82. The method of claim 78, wherein the AAV production host cells are infected with a helper virus after the culturing in the growth medium and before the culturing in the production medium, wherein the production medium is perfused 1 hour-6 hours after infection of the cells with the helper virus.

83. The method of any one of claims 51-77, wherein the AAV host production cells are cultured at the target cell density in a blend of growth and production media.

84. The method of claim 83, wherein the blend of growth and production media is a 10/90, 20/80, 30/70, 40/60, 50/50, 60/40, 70/30, 80/20, or a 90/10 blend of growth and production media.

85. The method of any one of claims 51-84, comprising culturing the AAV production host cells at the target cell density for about 48 hours, and/or about 2 days to about 14 days.

86. The method of claim 85, comprising culturing the AAV production host cells at the target cell density for a duration of about 48 hours, about 2 days, about 3 days, about 4 days, about 5 days, about 6 days, about 7 days, about 8 days, about 9 days, about 10 days, about 11 days, about 12 days, about 13 days, or about 14 days.

87. The method of claim 85 or 86, wherein the rAAV is released from the AAV production host cells into the supernatant of the production medium.

88. The method of claim 82, wherein the rAAV is predominantly released beginning after about 48 hours of infection of the cells with the helper virus.

89. The method of claim 88, wherein the perfusion is stopped at about 48 hours after infection.

90. The method of any one of claims 51-89, further comprising harvesting the rAAV.

91. The method of claim 90, wherein harvesting the rAAV is performed under batch mode conditions.

92. The method of any one of claims 51-91, further comprising downstream processing of the rAAV.

93. The method of claim 92, wherein the downstream processing comprises purifying, clarifying, and/or concentrating the rAAV.

94. The method of claim 93, wherein the downstream processing comprises filtering cellular debris, colloids, or aggregates.

95. The method of claim 94, wherein the filtering removes particles of cellular debris, colloids, or aggregates more than 1 μm in size.

96. The method of any one of claims 93-95, comprising purifying the rAAV using anion exchange (AEX) chromatography.
